(19) United States
(12) Patent Application Publication
(10) Pub. No.: US 2026/0224705 A1
(43) Pub. Date: Aug. 6, 2026

(54) COMPOSITIONS AND METHODS RELATING TO INHALABLE THERAPEUTIC COMPOSITIONS

(71) Applicant: North Carolina State University, Raleigh, NC (US)

(72) Inventors: Ke Cheng, Raleigh, NC (US); Xuan Mei, Raleigh, NC (US)

(21) Appl. No.: 19/151,156

(22) PCT Filed: Jan. 25, 2024
(86) PCT No.: PCT/US2024/012952

§ 371 Date: Jul. 25, 2025

Related U.S. Application Data
(60) Provisional application No. 63/481,486, filed on Jan. 25, 2023.

(51) Int. Cl. A61K 47/32; A61K 9/00; A61K 47/42; A61P 31/12; A61P 37/00

(52) U.S. Cl. CPC A61K 47/32; A61K 9/0073; A61K 47/42; A61P 31/12; A61P 37/00

(57) ABSTRACT

The present disclosure provides compositions and methods related to inhalable therapeutics. In particular, the present disclosure provides hydrogel-based inhalable bioadhesives that safely and effectively coat the airway to provide a barrier against pathogenic organisms, allergens, and environmental pollutants.

### CROSS REFERENCE TO RELATED APPLICATIONS

This application claims priority to and the benefit of U.S. Provisional Patent Application No. 63/481,486 filed Jan. 25, 2023, which is incorporated herein by reference in its entirety and for all purposes.

### FIELD

The present disclosure provides compositions and methods related to inhalable therapeutics. In particular, the present disclosure provides hydrogel-based inhalable bioadhesives that safely and effectively coat the airway to provide a barrier against pathogenic organisms, allergens, and environmental pollutants.

### BACKGROUND

After its emergence in late 2019, severe acute respiratory syndrome coronavirus 2 (SARS-CoV-2) has caused more than 600 million infections and more than 6.59 million deaths resulting from COVID-19 worldwide. Since viruses constantly change through mutation, several variants of the virus are currently creating the variant of concern (VOC). The emergence of new variants, especially the B.1.617.2 (Delta) and B.1.1.529 (Omicron), has driven new waves of sharp increases in COVID-19 cases all over the world. Vaccines, one of the effective strategies to protect people against infectious diseases by reducing morbidity and mortality, work primarily by producing neutralizing antibodies specific for the surface S protein. However, mutation of the S protein may possibly limit the efficiency of these vaccines. Thus, novel strategies that could effectively prevent the infection of SARS-CoV-2, ideally before it even reaches the target lung cells, are desperately needed.

Spread through airborne transmission, SARS-CoV-2 must initially penetrate the mucus and enter the cells lining the respiratory tract. Possible reasons for the high transmissibility of SARS-CoV-2 include an active viral replication in airway epithelia at an early stage of infection. As the first and top layer of the immune system, physical barriers such as the mucus lining the airways plays a key role in the defense against infections. The mucus barrier offers protection in two steps: first to trap the foreign pathogens and then to eliminate them through mucociliary clearance. Human airway mucus has played an important role in preventing many respiratory viruses from reaching target cells; however, impaired mucociliary clearance was found for SARS-CoV-2, which promotes viral spread in the respiratory tree to increase the risk of infections. The interaction between mucus and bioadhesive polymers (such as chitosan) have been studied, opening possibilities for mucus engineering to block SARS-CoV-2 infection, as well as other respiratory conditions.

### SUMMARY

Embodiments of the present disclosure include an inhalable bioadhesive composition comprising polyacrylic acid, or a derivative thereof; acrylic acid N-hydroxysuccinimide ester; and a mixture of water-soluble peptides. In some embodiments, the composition forms a cross-linked hydrogel.

In some embodiments, the composition is formulated as a plurality of microparticles. In some embodiments, the plurality of microparticles are from about 0.1 μm to about 100 μm. In some embodiments, the plurality of microparticles are from about 1.0 μm to about 50 μm. In some embodiments, the plurality of microparticles have an aerodynamic diameter that is from about 0.5 μm to about 5.0 μm.

In some embodiments, the composition forms the cross-linked hydrogel upon exposure to lung tissue. In some embodiments, the volume of the composition increases at least 5 times upon exposure to lung tissue.

In some embodiments, the mixture of water-soluble peptides comprises gelatin chitosan, extracellular matrix, and/or hypromellose. In some embodiments, the composition is formulated as a powder. In some embodiments, the water-soluble peptides are present in the composition at a concentration ranging from about 1% wt/vol to about 10% wt/vol. In some embodiments, the polyacrylic acid is present in the composition at a concentration ranging from about 10% vol/vol to about 20% vol/vol. In some embodiments, the acrylic acid N-hydroxysuccinimide ester is present in the composition at a concentration ranging from about 0.1% wt/vol to about 5.0% wt/vol. In some embodiments, the composition consists essentially of polyacrylic acid, acrylic acid N-hydroxysuccinimide ester, and gelatin.

In some embodiments, the composition further comprises a pharmaceutically acceptable carrier or excipient.

Embodiments of the present disclosure also include a method of preparing inhalable bioadhesive microparticles. In accordance with these embodiments, the method includes preparing an aqueous solution comprising polyacrylic acid, acrylic acid N-hydroxysuccinimide ester, and a mixture of water-soluble peptides; adding the aqueous solution to an oil in a dropwise manner to generate an emulsion; incubating the emulsion at a temperature ranging from about 10° C. to about 15° C. to generate a plurality of microparticles; and collecting the plurality of microparticles.

In some embodiments, the method further comprises heating and/or agitating the aqueous solution.

In some embodiments, the oil is a food grade oil.

In some embodiments, the plurality of microparticles are collected using filtration.

In some embodiments, the method further comprises washing the plurality of microparticles after collection.

In some embodiments, the method further comprises drying the plurality of microparticles.

In some embodiments, the method further comprises formulating the plurality of microparticles into a powder.

In some embodiments, the water-soluble peptides are present in the solution at a concentration ranging from about 1% wt/vol to about 10% wt/vol. In some embodiments, the polyacrylic acid is present in the solution at a concentration ranging from about 10% vol/vol to about 20% vol/vol. In some embodiments, the acrylic acid N-hydroxysuccinimide ester is present in the solution at a concentration ranging from about 0.1% wt/vol to about 5.0% wt/vol.

Embodiments of the present disclosure also include a method of reducing the likelihood of a pulmonary condition in a subject. In accordance with these embodiments, the method comprises administering any of the compositions described herein to the subject.

In some embodiments, the composition is administered as a powder using a dry powder inhalation device. In some embodiments, the composition is administered intratracheally.

In some embodiments, the composition forms a cross-linked hydrogel upon exposure to lung tissue, thereby forming a barrier that reduces the likelihood of the pulmonary condition in the subject.

In some embodiments, the composition is administered at a dose ranging from about 1-5 mg per kg of the subject's body weight.

In some embodiments, the pulmonary condition comprises a viral infection. In some embodiments, the virus is a respiratory virus. In some embodiments, the respiratory virus is selected from the group consisting of an adenovirus, a coronavirus, an influenza virus, a parainfluenza virus, a parvovirus, a respiratory syncytial virus, or a rhinovirus. In some embodiments, the pulmonary condition comprises an allergy or allergic disease. In some embodiments, the pulmonary condition comprises silicosis.

In some embodiments, reducing the likelihood of the viral infection comprises reducing infection rate.

In some embodiments, the composition reduces the likelihood of the pulmonary condition for at least 8 hours.

In some embodiments, the composition is administered prophylactically.

In some embodiments, the pulmonary condition is caused by exposure to an environmental pollutant or contaminant.

In some embodiments, the pulmonary condition is caused by an allergen. In some embodiments, the allergen comprises pollen. In some embodiments, the allergen comprises silica.

### BRIEF DESCRIPTION OF THE DRAWINGS

FIGS. 1A-1H: Fabrication and characterization of SHIELD. a, Schematic showing the concept of SHIELD. The process includes Inhalation (I), Swelling (II) and Adhesion (III). b, A representative SEM image showing the morphology of SHIELD particles before swelling. c, Aerodynamic diameter of SHIELD particles. Data are mean±SD. n=4 independent experiments. d-f, Swelling behavior study. (d), Representative optical microscopy images showing the morphological changes of SHIELD during a period of ten minutes in water. Scale, 50 μm. (e), Volume change of spherical hydrogel in contact with water. The volume was calculated from the measured diameters during the swelling process. Data are mean±SD. n=5 independent experiments. (f), a representative SEM image showing the hydrogel network after SHIELD swelling. g, FTIR spectrum showing the presence of PAAc-NHS ester within SHIELD particles. h, Mucoadhesive study using NH₂-labelled beads. Fluorescence images showing the interaction between microbeads (blue) and swollen SHIELD (red). In b and h, independent experiments were performed (n=5) with similar results.

FIGS. 2A-2G: Interaction between SHIELD and mucus. a, Optical images and SEM images showing control mucus and mucus+SHIELD after cryodesiccation. b, ¹H NMR spectra for mucus, SHIELD and mucus+SHIELD. c, Frequency sweep of the elastic (G′) and viscous modulus (G″) of (1) mucus, (2) mucus+SHIELD (10 mg/ml) and (3) mucus+SHIELD (20 mg/ml). Data are mean±SD. n=3 independent experiments. d, Summary of average G′ and G″ (at a frequency of 1.0 Hz). (1) mucus, (2) mucus+SHIELD (10 mg/ml) and (3) mucus+SHIELD (20 mg/ml). Data are mean±SD. P<0.05 was considered statistical significance. Statistical analysis was performed with ordinary one-way ANOVA with Tukey's multiple comparisons, n=3 independent experiments. e, Representative trajectories of particles within mucus and mucus+SHIELD at the same time scale. The trajectories were shifted according to their mean position. f, Ensemble-averaged geometric mean square displacements (MSD) as a function of time scale. n=48 for control group; n=67 for SHIELD group. g, The surface morphology change of pig tracheal tube before and after the direct spray of SHIELD. The swelling reaction of SHIELD was stopped by dipping the samples into 100% ethanol (30 s) for dehydration after 10 s or 10 min. Scale bar, 10 μm. In a and g, independent experiments were performed (n=3) with similar results.

FIGS. 3A-3J: SHIELD inhalation blocks the entry of SARS-CoV-2 pseudoviruses in a mouse model. a, A schematic showing the animal study design. Created with BioRender.com. b, Representative ex vivo IVIS imaging of the main organs from mice at different time points. Data are mean±SD. n=3 animals per group. (1) Lung, (2) Heart, (3) Liver, (4) Spleen, (5) Kidney. Corresponding quantification of fluorescence from lung on the right. c and d, H&E staining (left) and fluorescence image (right) showing the lining of SHIELD-formed hydrogel on the airway: c, bronchus; d, small bronchioles. Blue, DAPI stained nuclei; Red, Cy7 labelled SHIELD. Scale bars, 100 μm. Lungs were harvested from mice 4 h after inhalation. Pseudovirus did not replicate but only delivered a genetically encoded fluorescent reporter, of which the fluorescence signal can be detected by IVIS. e and h, Representative ex vivo IVIS imaging of lungs from mice challenged with SARS-CoV-2 pseudovirus with (e) D614G mutations and (h) D614G, E484K, N501Y, and K417N mutations. SHIELD was inhaled at 4 h, 8 h, or 24 h before challenge. The graphs below show the quantitative infection rates of pseudovirus (left) and anti-pseudovirus efficiency of SHIELD (right) at different time points after inhalation. The infection rate of pseudovirus was calculated by the radiant efficiency (p/s/cm²/sr)/(μW/cm²) from the infected area divided by that from the whole lung. The protective efficiency of each group was calculated as: (Infection rate_{Control}−Infection_{rate4h/8h/24h})/Infection rate_{Control}×100%. Data are shown as mean±SD, n=3 animals per group. f and i, Representative confocal images of pseudovirus in lung tissue from mice challenged with SARS-CoV-2 pseudovirus with (f) D614G mutations and (i) D614G, E484K, N501Y, and K417N mutations 8 h after SHIELD inhalation. Scale bar, 50 μm. g and j, Representative images of SARS-S immunohistochemistry staining in lung tissue from mice challenged with SARS-CoV-2 pseudovirus with (g) D614G mutations and (j) D614G, E484K, N501Y, and K417N mutations 8 h after SHIELD inhalation. Scale bar, 50 μm. The graphs show the quantitative analysis. Data are shown as mean±SD, n=3 animals per group. Statistical analysis was performed by two tailed unpaired t test. In c, d, f and i, independent experiments were performed (n=3) with similar results.

FIGS. 4A-4I: SHIELD inhalation protects African green monkeys from SARS-CoV-2 infection. a, Schematic depicting the nonhuman primate study design. Created with BioRender.com. b, Viral sgRNA copies/swab in NSs and BAL at various time points following challenge. Each dot represents data from one animal. n=3 animals per group. *p<0.05. Statistical analysis was performed by 2-way ANOVA. c, Viral loads calculated based on area under the curve (AUC) from control or SHIELD-protected animals. Data are mean±SD. n=3 per group. d, Representative H&E images of fixed lung tissues from SARS-CoV-2-infected African green monkeys (AGMs). At least seven images were taken per animal. Scale bars, 500 μm. Quantification of lung fibrosis of infected AGMs by Ashcroft scoring; seven tissue slices were analyzed for each animal. Data are shown as mean±SD, n=21 per group. *p<0.05. Statistical analysis was performed by two-tailed Mann-Whitney Test. Ashcroft scoring was performed blindly. e, Representative images of SARS-N IHC staining in fixed lung tissues from SARS-CoV-2-infected AGMs 7 d post-viral challenging. n=3 animals per group. Seven images were taken for each animal. Scale bars, 100 μm. f, Quantitation of positive SARS-N numbers in lung tissues of infected AGMs. Data are shown as mean±SD, n=21 per group. *p<0.05. Statistical analysis was performed by two-tailed Mann-Whitney Test. g, Representative images of RNAscope in situ hybridization detection of vRNA in infected AGMs. n=3 animals per group. Seven images were taken for each animal. Scale bar, 100 μm. Corresponding quantifications are shown on the right, n=21 biologically independent samples. Data are mean±SD. *p<0.05. Statistical analysis was performed by two-tailed Mann-Whitney Test. h and i, Representative immunofluorescence images of SARS-N(greyscale), CD206 (green) and DAPI (blue) for WA1 (h) or B.1.617.2 (Delta) (i) challenged animals. Scale bar, 100 μm. n=3 animals per group. Seven images were taken for each animal. Corresponding quantifications are shown on the right, n=21 biologically independent samples. Data are mean±SD. *p<0.05. Statistical analysis was performed by two-tailed Mann-Whitney Test.

FIG. 5: Schematic showing the reaction and structure of SHIELD microspheres.

FIGS. 6A-6B: Swelling behavior study in bronchoalveolar lavage (BAL) fluid. a, Representative optical microscopy images showing the morphological changes of SHIELD particles during a period of ten seconds in BAL fluid. Scale bar, 10 μm. b, Volume change of spherical hydrogel in contact with BAL fluid. Data are mean±SD. n=5 independent samples.

FIGS. 7A-7B: Swelling behavior study of labelled SHIELD particles. a, Fluorescence microscopy images showing the morphological changes of SHIELD particles during a period of 5 seconds in water. b, The morphology of SHIELD ten minutes after swelling. Independent experiments were performed (n=3) with similar results.

FIG. 8: Fluorescence stability of labelled SHIELD in a 72 h period. Data are mean±SD. n=5 biologically independent samples.

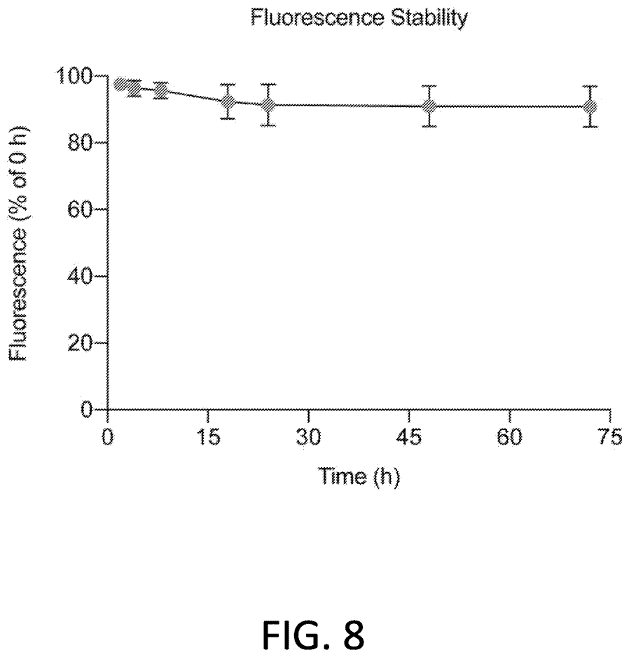

FIGS. 9A-9B: Adsorption of mucin to SHIELD particles. a, Calibration curve of O.D. towards mucin with different concentrations. b, Adsorption of mucin to SHIELD. Data are mean±SD. n=5 independent experiments.

FIGS. 10A-10C: SHIELD interacting with mucus. a, Red, SHIELD; b, Green, wheat germ agglutinin-stained mucus; c, Merged image. Independent experiments were performed (n=3) with similar results.

FIG. 11: SHIELD interacting with mucus in 3D airway model. Blue, nuclear stained with DAPI. Green, wheat germ agglutinin-stained mucus. Red, Cy7 labelled SHIELD. Scale bar, 100 μm. Independent experiments were performed (n=3) with similar results.

FIG. 12: Migration of particles in different media. The deposition of particles in different media (mucus and mucus+SHIELD) at different time points (0 min, 5 min and 15 min). Deposition was tracked by confocal microscope using 3D mode. Independent experiments were performed (n=3) with similar results.

FIGS. 13A-13D: SHIELD offers protection against air-borne particles. a, Illustration of the experimental process. Nebulizer was used to generate aerosol containing polystyrene fluorescent beads. Representative trajectories of particles in (b) mucus and (c) mucus+SHIELD for 20 s. The trajectories were shifted according to their mean position. d, Ensemble-averaged geometric mean square displacements (MSD) as a function of time. n=49 for control group; n=57 for SHIELD group.

FIGS. 14A-14C: Inhaler device for the mouse studies. a, The overview of the inhaler. The enlarged image shows the air flow for powder. b, A hole was bored in the apex of the taper to admit the air. c, The cap of the 2 ml centrifuge tube served as powder bed.

FIG. 15: The luminescence/fluorescence signal of SHIELD in IVIS. Representative ex vivo IVIS imaging of lungs from mice at different time points. Three different channels (Cy 7, GFP, and luminescence) were included for each lung. n=3 animals per group.

FIG. 16: Fluorescence emission spectrum of FRET assay. The excitation for Q610 was at 570 nm and for DiD was at 630 nm. FRET occurred between Q610 and DiD on H1N1 virus particles. Little emission signal was observed for DiD at Q610 excitation.

FIG. 17: Confocal image of lung tissue showing the entry of H1N1 virus into cells. After DiD labelled virus (Magenta) challenging, lung tissues were harvested and stained with anti-HA antibody (Green). The co-localization of anti-HA antibody and DiD confirms the virus particles entering the cells. Independent experiments were performed (n=5) with similar results.

FIGS. 18A-18C: SHIELD inhalation inhibits the infection of PVM viruses in a mouse model. a, Representative ex vivo IVIS imaging of lungs from mice challenged with PVM. SHIELD was inhaled at 4 h, 8 h, or 24 h before challenge. b, Quantitative anti-infection efficiency. Data are shown as mean±SD, n=3 animals per group. c, Representative confocal images showing pseudovirus in lung tissue from mice challenged with PVM 8 h after SHIELD inhalation. The graphics show the semiquantitative analysis. Scale bar, 50 μm. Data are shown as mean±SD, n=3 animals per group.

FIGS. 19A-19B: SHIELD inhalation inhibits the entry of H1N1 viruses in a mouse model. a, Representative ex vivo IVIS imaging of lungs from mice challenged with H1N1 virus. SHIELD was inhaled at 4 h, 8 h, or 24 h before challenge. b, Quantitative anti-infection efficiency. Data are shown as mean±SD, n=3 animals per group.

FIG. 20: RNA level of virus shedding isolated from nasal swabs. All the mice were challenged with PVM (with GFP) for 24 h. Nasal swabs were collected from mice after treatment with SHIELD for 4 h, 8 h, and 24 h. PCR test was performed using GFP primers. Data are mean±SD. n=5 animals per group. Statistical analysis was performed by two-sided unpaired t test.

FIGS. 21A-21B: Biocompatibility of SHIELD in human bronchial epithelial cells. a, The viability of cells cultured with SHIELD for three days. n=6 biologically independent samples. Data are mean±SD. b, live/dead staining of cells after three days cultured with SHIELD. Scale bar, 200 μm.

FIGS. 22A-22E: Toxicity study on repeated SHIELD dosing. a-d Evaluation of lung functions in mice with various regimens of SHIELD dosing. Baseline reads were recorded before inhalation and all the results were presented as percentages of baseline. (a) pulmonary inspiratory capacity; (b) respiratory elastance; (c) hysteresis area; (d) forced expiratory volume (FEV) to forced vital capacity (FVC) ratio. n=3 biologically independent animals. Data are mean±SD. e, H&E staining of major organs harvested from control group and SHIELD-treated group (inhalation for 14 consecutive days). Scale bar, 500 μm. Independent experiments were performed (n=3) with similar results.

FIGS. 23A-23D: Effects of repeated SHIELD inhalation on tracheas and bronchus. H&E staining showing the morphologies of (a) trachea tube, and (b)-(d) bronchus harvested from mice treated with SHIELD for 14 consecutive days. Scale bar, 500 μm. Independent experiments were performed (n=3) with similar results.

FIGS. 24A-24B: Pulmonary mucociliary clearance test. a, Baseline deposition. b, Pulmonary MCC elimination (% baseline deposition). Carboxylate-modified microspheres with yellow-green fluorescence (2 μm diameter) were used for in vivo MCC test. Data are mean±SD. *p<0.05; ns, not significant. n=4 biologically independent experiments per group. Statistical significance was determined by a one-way ANOVA and Tukey's multiple comparison test.

FIGS. 25A-25B: Evaluation of slgA level in mouse BAL fluid. a, Calibration curve on O.D. values vs. different sIgA concentrations. b, Concentrations of sIgA in BAL fluid collected from control mice and mice with 2 weeks of SHIELD inhalation. Data are mean±SD. n=3 animals per group. *p<0.05; ns, no significance. Statistical analysis was performed by two-tailed Mann-Whitney Test.

FIGS. 26A-26B: The effects of SHIELD on RNA extraction and PCR. (a) Viral sgRNA copies/ml of SHIELD in different concentrations (0.01, 0.05, 0.1, 0.5 and 1 mg/ml). ND, not detected. The limit of quantification (LOQ) for this assay is approximately 31 RNA cp/mL (1.49 log 10) with 800 μL of sample. n=5 per group. (b) Viral sgRNA results of four BAL samples mixed with or without SHIELD (0.05 mg/ml). Data are mean±SD. *p<0.05; n=3 per group. Statistical analysis was performed by two-way ANOVA and Bonferroni multiple comparison test.

FIGS. 27A-27B: Body weight changes after SARS-CoV-2 challenge in AGMs. a, AGMs challenged with WA1. b, AGMs challenged with B.1.617.2 (Delta) variant. n=3 per group.

FIGS. 28A-28B: Temperature changes after SARS-CoV-2 challenge in AGMs. a, AGMs challenged with WA1. b, AGMs challenged with B.1.617.2 (Delta) variant. n=3 per group.

FIG. 29: Hematology analysis of AGMs after SHIELD protection. Data are mean±SD. n=3 animals per group.

FIGS. 30A-30F: Fabrication and characterization of bioadhesive microspheres. a, Schematic image shows the airway protection by inhalable bioadhesive microsphere. b, SEM images of different sized microspheres. Scale bar, 5 μm (up left), 5 μm (up right), 50 μm (down left), 50 μm (down right). c, Enlarged SEM image of bioadhesive microsphere. Scale bar, 10 μm. d, Confocal fluorescent image of cy5 labeled bioadhesive microsphere. Scale bar, 20 μm. e, Energy dispersive spectrum of bioadhesive microsphere. f, Elemental mapping of bioadhesive microsphere to show the carbon, nitrogen and oxygen distribution. Scale bar, 10 μm.

FIGS. 31A-31G: Swelling behavior of bioadhesive microsphere and hydrogel formation. a, Schematic image to show the swelling behavior of bioadhesive microsphere and its chemical mechanism. b, Morphological changes of bioadhesive microsphere in pure water by optical microscope. Scale bar, 100 μm. c, Volume changes of 15 μm bioadhesive microsphere in ddH₂O, normal saline and PBS buffer. d, Volume changes of different sized bioadhesive microsphere in ddH₂O. e, Schematic image to show the swelling process of bioadhesive microsphere and hydrogel formation. f-g, SEM image (f) and confocal fluorescent images (g) to show the morphology of dry microsphere, swelling microsphere and hydrogel. Scale bar, 20 μm.

FIGS. 32A-32H: Allergen isolation by bioadhesive hydrogel. a, Confocal fluorescent image of RhB labeled pollen particles. Scale bar, 100 μm. b, 3D imaging of RhB labeled pollen (yellow) on cy5 labeled hydrogel (red). c, Confocal fluorescent images to show the pollen-hydrogel interface. Scale bar, 200 μm. d, Western blot to show the penetrated pollen allergen protein in the lower PBS solution. The bioadhesive microspheres or Nasaleze formed a thick layer (left) or thin layer (middle) hydrogel to isolate the pollen particles, or directly mixed (right) with pollen particles. e, Confocal fluorescent image of RhB labeled silica particles. Scale bar, 50 μm. f, 3D imaging of RhB labeled silica (yellow) on cy5 labeled hydrogel (red). g, Confocal fluorescent images to show the silica-hydrogel interface. Scale bar, 200 μm. h, 3D confocal fluorescent images to show the isolation of dust and HBE cells by bioadhesive hydrogel. Red, cy5 labeled dust; yellow, RhB labeled hydrogel; blue, cell nucleus.

FIGS. 33A-33C: Bioadhesive hydrogel formation in vivo. a, Fluorescent images to show the hydrogel formation by bioadhesive microsphere on mouse bronchi. b, Fluorescent images showing the hydrogel formation by bioadhesive microsphere on mouse nasal cavity. c, Bioadhesive hydrogel formation in porcine airways. The proportion of these images are the same. Scale bar, 2 mm.

FIGS. 34A-34D: Pollen allergy prevention by bioadhesive hydrogel in vivo. a, The experimental study schematic of the pollen allergy prevention mice model. b, IVIS imaging of the mouse nasal cavity after bioadhesive microsphere and pollen inhalation. c, Confocal fluorescent images to show the pollen prevention by hydrogel on mouse nasal mucosa. Scale bar, 100 μm. d, Serum IgE and cytokines expression in normal mice, pollen treated mice, and bioadhesive microsphere pre-inhalation mice.

FIGS. 35A-35G: Silicosis prevention by bioadhesive hydrogel in vivo. a, The experimental study schematic of the silica prevention mice model. b, Confocal fluorescent images to show the pollen prevention by hydrogel on mouse trachea. c, H&E staining of lung of normal mice, silica-treated mice, and bioadhesive microsphere pre-inhalation mice. d-g, Quantification of lung function measurements. d, pulmonary inspiratory capacity; e, pulmonary resistance; f, static lung compliance; g, elastic resistance.

### DETAILED DESCRIPTION

Embodiments of the present disclosure include compositions and methods related to inhalable therapeutics. In particular, the present disclosure provides hydrogel-based inhalable bioadhesives that safely and effectively coat the airway to provide a barrier against pathogenic organisms, allergens, and environmental pollutants.

As described further herein, inhalable bioadhesive compositions were developed (termed “SHIELD” for a spherical hydrogel inhalation for enhanced lung defense) to offer protection against respiratory insults, including but not limited to, infections from SARS-CoV-2 WA1 and the B.1.617.2 (Delta) variant. Fabricated from food grade materials, SHIELD is both effective and safe, without affecting normal lung functions or causing toxicity. Unlike vaccines, SHIELD enhances the diffusional barrier properties of mucus to provide physical protection against virus infection. Acting as an innate defense mechanism, the airway mucus layer plays a key role in trapping and keeping pathogens away from the host epithelial cell surface. The main component of mucus is mucins (0.2-5% w/w), which are glycoproteins with a protein core and carbohydrate side chains. To penetrate the mucus barrier, viruses have developed various strategies, such as secreting mucolytic enzymes or altering their surface properties. It has been reported that the SARS-CoV-2 virus infects both ciliated cells and secretory cells in human airway, resulting in its efficient upper airway transmission. The presence of ACE2 was confirmed in the motile cilia and ~80% of the human respiratory epithelium (from the nasal cavity down to the lower bronchus) is densely covered with cilia. This also provides a large surface area for SARS-CoV-2 to bind and enter host cells. Hence, it would be ideal to design a strategy to strengthen the mucosal barrier without affecting the normal mucociliary clearance (MCC) properties.

There are some studies demonstrating the mucus reinforcement properties of complexing polymer molecules. Low molar mass mucoadhesive polymers, such as chitosan, have shown potential in mucosal barrier enhancement and have been used to develop various vaginal and intestinal dosage formulations. Previous studies have demonstrated that various dietary compounds also alter mucus barrier properties, such as green tea polyphenol epigallocatechin gallate. Orally administered gelatin tannate protected the gastric mucus layer from breakdown to modulate the gut microbiota composition. These studies opened possibilities for mucus engineering; however, targeted in vivo delivery remains challenging, especially in the pulmonary system.

As demonstrated further herein, SHIELD was fabricated in the form of a dry powder, with a size between 1-5 μm. Such size facilitated its efficient deposition by inertial impaction and sedimentation deep into the airways and lung tissues. Taking advantage of dry powder inhalation (DPI), SHIELD is easy to handle for daily administration and only requires low-cost inhaler devices. SHIELD can serve as a supplement to face masks and/or as an alternative when face masks are not used (e.g., during eating and drinking, swimming, heavy excising, or for small children who are reluctant to wear masks).

The continued mutation of SARS-CoV-2 virus leads to the emergence of a series viral variants, causing the new rise in reported COVID-19 cases. The mutation in S-protein may impair the neutralizing epitopes, reduce vaccination efficiency, and increase virus transmission. New vaccines may be needed to combat the different variants. In contrast, the SHIELD strategy will not be limited by viral mutation. Physical protection, such as masks or other facial covering methods, has shown great effectiveness in reducing virus transmission during the pandemic. Embodiments of the present disclosure take advantage of mucus as a natural mechanical barrier against foreign pathogens. SHIELD particles reinforce the mucus structure and restrict the penetration of foreign pathogens. The broad-spectrum protection of SHIELD towards other respiratory pathogens was also confirmed in the present disclosure with inactivated H1N1 virus and PVM. SHIELD can offer effective and additional protection to the general population against COVID-19 and other respiratory pathogens and insults.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The terms “administration of” and “administering” a composition as used herein refers to providing a composition of the present disclosure to a subject in need of treatment (e.g., prophylactic treatment for a respiratory condition). The compositions of the present disclosure may be administered by oral, parenteral (e.g., intramuscular, intraperitoneal, intravenous, ICV, intracisternal injection or infusion, subcutaneous injection, nebulization, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In some embodiments, the composition is administered intratracheally.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition in the patient (e.g., viral infection or other respiratory condition). The compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

Formulations suitable for administration include, for example, aqueous sterile injection solutions, which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection or other respiratory insult). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease. As described further herein, “treatment” and “prevention” includes use of a hydrogel-based composition comprising a therapeutic agent for the treatment and/or prevention of a respiratory condition in a subject (e.g., a human subject), as well as use of a hydrogel-based composition comprising a therapeutic agent for the manufacture of a medicament to treat and/or prevent a respiratory condition.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, cardiovascular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. THERAPEUTIC COMPOSITIONS AND METHODS OF DELIVERY

As described further herein, embodiments of the present disclosure include an inhalable bioadhesive composition that includes polyacrylic acid, or a derivative thereof, acrylic acid N-hydroxysuccinimide ester, and a mixture of water-soluble peptides. In accordance with these embodiments, the composition forms a cross-linked hydrogel that provides a barrier that reduces or prevents the likelihood of an adverse condition from occurring. In some embodiments, the composition is administered to a subject. In some embodiments, the composition is formulated for administration to the respiratory tract of a subject. In some embodiments, the composition is administered intratracheally.

In some embodiments, administering the compositions of the present disclosure to the lungs of a subject facilitates the formation of a cross-linked hydrogel upon exposure to the lung tissue. In some embodiments, the cross-linked hydrogel swells and adheres to the lung tissue of a subject, thus creating a protective barrier that reduces or prevents the likelihood of developing a pulmonary condition. In some embodiments, the volume of the composition increases at least 5 times upon exposure to lung tissue. In some embodiments, the volume of the composition increases at least 6 times upon exposure to lung tissue. In some embodiments, the volume of the composition increases at least 7 times upon exposure to lung tissue. In some embodiments, the volume of the composition increases at least 8 times upon exposure to lung tissue. In some embodiments, the volume of the composition increases at least 9 times upon exposure to lung tissue. In some embodiments, the volume of the composition increases at least 10 times upon exposure to lung tissue.

In some embodiments, and as described further herein, the composition is formulated as a plurality of microparticles. In some embodiments, the plurality of microparticles are of a size that is about 0.1 μm to about 100 μm. In some embodiments, the plurality of microparticles are of a size that is about 1.0 μm to about 100 μm. In some embodiments, the plurality of microparticles are of a size that is about 5.0 μm to about 100 μm. In some embodiments, the plurality of microparticles are of a size that is about 25 μm to about 100 μm. In some embodiments, the plurality of microparticles are of a size that is about 50 μm to about 100 μm. In some embodiments, the plurality of microparticles are of a size that is about 0.1 μm to about 50 μm. In some embodiments, the plurality of microparticles are of a size that is about 0.1 μm to about 25 μm. In some embodiments, the plurality of microparticles are of a size that is about 0.1 μm to about 5.0 μm. In some embodiments, the plurality of microparticles are of a size that is about 0.1 μm to about 1.0 μm. In some embodiments, the plurality of microparticles are of a size that is about 0.1 μm to about 50 μm. In some embodiments, the plurality of microparticles are of a size that is about 1.0 μm to about 50 μm. In some embodiments, the plurality of microparticles are of a size that is about 0.1 μm to about 50 μm. In some embodiments, the plurality of microparticles are of a size that is about 0.1 μm to about 10 μm.

In some embodiments, the plurality of microparticles have an aerodynamic diameter that is from about 0.5 μm to about 5.0 μm. In some embodiments, the plurality of microparticles have an aerodynamic diameter that is from about 1.0 μm to about 5.0 μm. In some embodiments, the plurality of microparticles have an aerodynamic diameter that is from about 2.0 μm to about 5.0 μm. In some embodiments, the plurality of microparticles have an aerodynamic diameter that is from about 3.0 μm to about 5.0 μm. In some embodiments, the plurality of microparticles have an aerodynamic diameter that is from about 4.0 μm to about 5.0 μm. In some embodiments, the plurality of microparticles have an aerodynamic diameter that is from about 0.5 μm to about 4.0 μm. In some embodiments, the plurality of microparticles have an aerodynamic diameter that is from about 0.5 μm to about 3.0 μm. In some embodiments, the plurality of microparticles have an aerodynamic diameter that is from about 0.5 μm to about 2.0 μm. In some embodiments, the plurality of microparticles have an aerodynamic diameter that is from about 0.5 μm to about 1.0 μm.

In some embodiments, the mixture of water-soluble peptides comprises gelatin chitosan, extracellular matrix, and/or hypromellose. In some embodiments, the mixture of water-soluble peptides comprises gelatin. In some embodiments, the mixture of water-soluble peptides comprises chitosan. In some embodiments, the mixture of water-soluble peptides comprises extracellular matrix. In some embodiments, the mixture of water-soluble peptides comprises hypromellose. In some embodiments, the composition is formulated as a powder. In some embodiments, the water-soluble peptides are present in the composition at a concentration ranging from about 1% wt/vol to about 10% wt/vol. In some embodiments, the water-soluble peptides are present in the composition at a concentration ranging from about 1% wt/vol to about 7.5% wt/vol. In some embodiments, the water-soluble peptides are present in the composition at a concentration ranging from about 1% wt/vol to about 5% wt/vol. In some embodiments, the water-soluble peptides are present in the composition at a concentration ranging from about 1% wt/vol to about 2.5% wt/vol. In some embodiments, the water-soluble peptides are present in the composition at a concentration ranging from about 2.5% wt/vol to about 10% wt/vol. In some embodiments, the water-soluble peptides are present in the composition at a concentration ranging from about 5% wt/vol to about 10% wt/vol. In some embodiments, the water-soluble peptides are present in the composition at a concentration ranging from about 7.5% wt/vol to about 10% wt/vol. In some embodiments, the water-soluble peptides are present in the composition at a concentration of about 5% wt/vol.

In some embodiments, the polyacrylic acid is present in the composition at a concentration ranging from about 10% vol/vol to about 20% vol/vol. In some embodiments, the polyacrylic acid is present in the composition at a concentration ranging from about 10% vol/vol to about 17.5% vol/vol. In some embodiments, the polyacrylic acid is present in the composition at a concentration ranging from about 10% vol/vol to about 15% vol/vol. In some embodiments, the polyacrylic acid is present in the composition at a concentration ranging from about 10% vol/vol to about 12.5% vol/vol. In some embodiments, the polyacrylic acid is present in the composition at a concentration ranging from about 12.5% vol/vol to about 20% vol/vol. In some embodiments, the polyacrylic acid is present in the composition at a concentration ranging from about 15% vol/vol to about 20% vol/vol. In some embodiments, the polyacrylic acid is present in the composition at a concentration ranging from about 17.5% vol/vol to about 20% vol/vol. In some embodiments, the polyacrylic acid is present in the composition at a concentration of about 15% vol/vol.

In some embodiments, the acrylic acid N-hydroxysuccinimide ester is present in the composition at a concentration ranging from about 0.1% wt/vol to about 5.0% wt/vol. In some embodiments, the acrylic acid N-hydroxysuccinimide ester is present in the composition at a concentration ranging from about 0.5% wt/vol to about 5.0% wt/vol. In some embodiments, the acrylic acid N-hydroxysuccinimide ester is present in the composition at a concentration ranging from about 1.0% wt/vol to about 5.0% wt/vol. In some embodiments, the acrylic acid N-hydroxysuccinimide ester is present in the composition at a concentration ranging from about 2.5% wt/vol to about 5.0% wt/vol. In some embodiments, the acrylic acid N-hydroxysuccinimide ester is present in the composition at a concentration ranging from about 4.0% wt/vol to about 5.0% wt/vol. In some embodiments, the acrylic acid N-hydroxysuccinimide ester is present in the composition at a concentration ranging from about 0.1% wt/vol to about 4.0% wt/vol. In some embodiments, the acrylic acid N-hydroxysuccinimide ester is present in the composition at a concentration ranging from about 0.1% wt/vol to about 2.5% wt/vol. In some embodiments, the acrylic acid N-hydroxysuccinimide ester is present in the composition at a concentration ranging from about 0.1% wt/vol to about 1.0% wt/vol. In some embodiments, the acrylic acid N-hydroxysuccinimide ester is present in the composition at a concentration ranging from about 0.1% wt/vol to about 0.5% wt/vol. In some embodiments, the acrylic acid N-hydroxysuccinimide ester is present in the composition at a concentration of about 0.5% wt/vol.

In some embodiments, the composition consists essentially of polyacrylic acid, acrylic acid N-hydroxysuccinimide ester, and gelatin. In some embodiments, the composition consists of polyacrylic acid, acrylic acid N-hydroxysuccinimide ester, and gelatin. In some embodiments, the composition comprises gelatin at a concentration of about 5% wt/vol, polyacrylic acid at a concentration of about 15% vol/vol, and acrylic acid N-hydroxysuccinimide ester at a concentration of about 0.5% wt/vol.

In some embodiments, the composition further comprises at least one pharmaceutically acceptable excipient or carrier. A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

Embodiments of the present disclosure also include a method of preparing inhalable bioadhesive microparticles. In accordance with these embodiments, the method includes preparing an aqueous solution or composition comprising polyacrylic acid, acrylic acid N-hydroxysuccinimide ester, and a mixture of water-soluble peptides, adding the aqueous solution to an oil in a dropwise manner to generate an emulsion, incubating the emulsion at a temperature ranging from about 10° C. to about 15° C. to generate a plurality of microparticles, and collecting the plurality of microparticles. In some embodiments, the method further comprises heating and/or agitating the aqueous solution. In some embodiments, the oil is a food grade oil.

In some embodiments, the plurality of microparticles are collected using filtration. In some embodiments, the method further comprises washing the plurality of microparticles after collection. In some embodiments, the method further comprises drying the plurality of microparticles. In some embodiments, the method further comprises formulating the plurality of microparticles into a powder.

In some embodiments, methods of preparing the plurality of microparticles of the present disclosure result in a plurality of microparticles comprising a size that is about 0.1 μm to about 100 μm. In some embodiments, the plurality of microparticles are of a size that is about 1.0 μm to about 100 μm. In some embodiments, the plurality of microparticles are of a size that is about 5.0 μm to about 100 μm. In some embodiments, the plurality of microparticles are of a size that is about 25 μm to about 100 μm. In some embodiments, the plurality of microparticles are of a size that is about 50 μm to about 100 μm. In some embodiments, the plurality of microparticles are of a size that is about 0.1 μm to about 50 μm. In some embodiments, the plurality of microparticles are of a size that is about 0.1 μm to about 25 μm. In some embodiments, the plurality of microparticles are of a size that is about 0.1 μm to about 5.0 μm. In some embodiments, the plurality of microparticles are of a size that is about 0.1 μm to about 1.0 μm. In some embodiments, the plurality of microparticles are of a size that is about 0.1 μm to about 50 μm. In some embodiments, the plurality of microparticles are of a size that is about 1.0 μm to about 50 μm. In some embodiments, the plurality of microparticles are of a size that is about 0.1 μm to about 50 μm. In some embodiments, the plurality of microparticles are of a size that is about 0.1 μm to about 10 μm.

In some embodiments, methods of preparing the plurality of microparticles of the present disclosure result in a plurality of microparticles comprising an aerodynamic diameter that is from about 0.5 μm to about 5.0 μm. In some embodiments, the plurality of microparticles have an aerodynamic diameter that is from about 1.0 μm to about 5.0 μm. In some embodiments, the plurality of microparticles have an aerodynamic diameter that is from about 2.0 μm to about 5.0 μm. In some embodiments, the plurality of microparticles have an aerodynamic diameter that is from about 3.0 μm to about 5.0 μm. In some embodiments, the plurality of microparticles have an aerodynamic diameter that is from about 4.0 μm to about 5.0 μm. In some embodiments, the plurality of microparticles have an aerodynamic diameter that is from about 0.5 μm to about 4.0 μm. In some embodiments, the plurality of microparticles have an aerodynamic diameter that is from about 0.5 μm to about 3.0 μm. In some embodiments, the plurality of microparticles have an aerodynamic diameter that is from about 0.5 μm to about 2.0 μm. In some embodiments, the plurality of microparticles have an aerodynamic diameter that is from about 0.5 μm to about 1.0 μm.

In some embodiments of the method, the mixture of water-soluble peptides comprises gelatin chitosan, extracellular matrix, and/or hypromellose. In some embodiments, the mixture of water-soluble peptides comprises gelatin. In some embodiments, the mixture of water-soluble peptides comprises chitosan. In some embodiments, the mixture of water-soluble peptides comprises extracellular matrix. In some embodiments, the mixture of water-soluble peptides comprises hypromellose.

In some embodiments of the method, the water-soluble peptides are present in the solution at a concentration ranging from about 1% wt/vol to about 10% wt/vol. In some embodiments, the water-soluble peptides are present in the solution at a concentration ranging from about 1% wt/vol to about 7.5% wt/vol. In some embodiments, the water-soluble peptides are present in the solution at a concentration ranging from about 10% wt/vol to about 5% wt/vol. In some embodiments, the water-soluble peptides are present in the solution at a concentration ranging from about 1% wt/vol to about 2.5% wt/vol. In some embodiments, the water-soluble peptides are present in the solution at a concentration ranging from about 2.5% wt/vol to about 10% wt/vol. In some embodiments, the water-soluble peptides are present in the solution at a concentration ranging from about 5% wt/vol to about 10% wt/vol. In some embodiments, the water-soluble peptides are present in the solution at a concentration ranging from about 7.5% wt/vol to about 10% wt/vol. In some embodiments, the water-soluble peptides are present in the solution at a concentration of about 5% wt/vol.

In some embodiments of the method, the polyacrylic acid is present in the solution at a concentration ranging from about 10% vol/vol to about 20% vol/vol. In some embodiments, the polyacrylic acid is present in the solution at a concentration ranging from about 10% vol/vol to about 17.5% vol/vol. In some embodiments, the polyacrylic acid is present in the solution at a concentration ranging from about 10% vol/vol to about 15% vol/vol. In some embodiments, the polyacrylic acid is present in the solution at a concentration ranging from about 10% vol/vol to about 12.5% vol/vol. In some embodiments, the polyacrylic acid is present in the solution at a concentration ranging from about 12.5% vol/vol to about 20% vol/vol. In some embodiments, the polyacrylic acid is present in the solution at a concentration ranging from about 15% vol/vol to about 20% vol/vol. In some embodiments, the polyacrylic acid is present in the solution at a concentration ranging from about 17.5% vol/vol to about 20% vol/vol. In some embodiments, the polyacrylic acid is present in the solution at a concentration of about 15% vol/vol.

In some embodiments of the method, the acrylic acid N-hydroxysuccinimide ester is present in the solution at a concentration ranging from about 0.1% wt/vol to about 5.0% wt/vol. In some embodiments, the acrylic acid N-hydroxysuccinimide ester is present in the solution at a concentration ranging from about 0.5% wt/vol to about 5.0% wt/vol. In some embodiments, the acrylic acid N-hydroxysuccinimide ester is present in the solution at a concentration ranging from about 1.0% wt/vol to about 5.0% wt/vol. In some embodiments, the acrylic acid N-hydroxysuccinimide ester is present in the solution at a concentration ranging from about 2.5% wt/vol to about 5.0% wt/vol. In some embodiments, the acrylic acid N-hydroxysuccinimide ester is present in the solution at a concentration ranging from about 4.0% wt/vol to about 5.0% wt/vol. In some embodiments, the acrylic acid N-hydroxysuccinimide ester is present in the solution at a concentration ranging from about 0.1% wt/vol to about 4.0% wt/vol. In some embodiments, the acrylic acid N-hydroxysuccinimide ester is present in the solution at a concentration ranging from about 0.1% wt/vol to about 2.5% wt/vol. In some embodiments, the acrylic acid N-hydroxysuccinimide ester is present in the solution at a concentration ranging from about 0.1% wt/vol to about 1.0% wt/vol. In some embodiments, the acrylic acid N-hydroxysuccinimide ester is present in the solution at a concentration ranging from about 0.1% wt/vol to about 0.5% wt/vol. In some embodiments, the acrylic acid N-hydroxysuccinimide ester is present in the solution at a concentration of about 0.5% wt/vol.

Embodiments of the present disclosure also include a method of reducing the likelihood of a pulmonary condition in a subject. In accordance with these embodiments, the method includes administering any of the compositions described herein to the subject. In some embodiments, the composition forms a cross-linked hydrogel upon exposure to lung tissue, thereby forming a barrier that reduces the likelihood of the pulmonary condition in the subject.

In some embodiments, the composition is administered as a powder using a dry powder inhalation device. In some embodiments, the composition is administered intratracheally. In some embodiments, the composition is administered at a dose ranging from about 1-5 mg per kg of the subject's body weight. In some embodiments, the composition reduces the likelihood of the pulmonary condition for at least 8 hours. In some embodiments, the composition reduces the likelihood of the pulmonary condition for at least 12 hours. In some embodiments, the composition reduces the likelihood of the pulmonary condition for at least 16 hours. In some embodiments, the composition reduces the likelihood of the pulmonary condition for at least 20 hours. In some embodiments, the composition reduces the likelihood of the pulmonary condition for at least 24 hours. In some embodiments, the composition reduces the likelihood of the pulmonary condition for at least 36 hours. In some embodiments, the composition reduces the likelihood of the pulmonary condition for at least 48 hours.

In some embodiments, the composition is administered prophylactically to a subject, and the administration of the composition reduces or prevents a pulmonary condition in a subject. In some embodiments, the pulmonary condition comprises a viral infection. In some embodiments, the virus is a respiratory virus. In some embodiments, the respiratory virus is selected from the group consisting of an adenovirus, a coronavirus, an influenza virus, a parainfluenza virus, a parvovirus, a respiratory syncytial virus, or a rhinovirus. In some embodiments, reducing the likelihood of the viral infection comprises reducing infection rate.

In some embodiments, the pulmonary condition is caused by exposure to an environmental pollutant or contaminant. In some embodiments, the pulmonary condition is caused by an allergen. In some embodiments, the allergen comprises pollen. In some embodiments, the allergen comprises silica. In some embodiments, the pulmonary condition comprises an allergy or allergic disease. In some embodiments, the pulmonary condition comprises silicosis.

For any compositions described herein comprising the microparticles, a therapeutically effective amount can be initially determined from animal models. A therapeutically effective dose can also be determined from human data which are known to exhibit similar pharmacological activities, such as other adjuvants. Higher doses may be required for parenteral administration. The applied dose can be adjusted based on the relative bioavailability and potency of the administered microparticles. Adjusting the dose to achieve maximal efficacy based on the methods described above and other methods as are well-known in the art is well within the capabilities of the ordinarily skilled person in the art.

The various compositions of the present disclosure provide dosage forms, formulations, and methods that confer advantages and/or beneficial pharmacokinetic profiles. A composition of the disclosure can be utilized in dosage forms in pure or substantially pure form, in the form of its pharmaceutically acceptable salts, and also in other forms including anhydrous or hydrated forms. A beneficial pharmacokinetic profile may be obtained by administering a formulation or dosage form suitable for once, twice a day, or three times a day, or more administration comprising one or more composition of the disclosure present in an amount sufficient to provide the required concentration or dose of the composition to an environment of use to treat a disease disclosed herein, in particular a cancer.

A medicament or treatment of the disclosure may comprise a unit dosage of at least one composition of the disclosure to provide therapeutic effects. A “unit dosage or “dosage unit” refers to a unitary (e.g., a single dose), which is capable of being administered to a patient, and which may be readily handled and packed, remaining as a physically and chemically stable unit dose comprising either the active agents as such or a mixture with one or more solid or liquid pharmaceutical excipients, carriers, or vehicles.

### 3. MATERIALS AND METHODS

All experiments were performed in accordance with relevant guidelines and ethical regulations approved by the North Carolina State University.

Fabrication of SHIELD particles. To prepare SHIELD particles, acrylic acid (147230, Sigma-Aldrich, USA), acrylic acid N-hydroxysuccinimide ester (AAc-NHS ester, A8060, Sigma-Aldrich, USA), and gelatin (G1890, Sigma-Aldrich, USA) were used. SHIELD particles were prepared using a water-in-oil emulsion method. lg Gelatin was dissolved in 20 ml ultrapure water with 3 ml acrylic acid. This solution was warmed to 45° C., followed by the addition of 100 mg AAc-NHS ester. The mixture was stirred at 45° C. for 20 min and sonicated for 30 s to make sure all the AAc-NHS ester was dissolved. Due to the electrostatic interaction, acrylic acid monomer and AAc-NHS ester are bound to the gelatin chain, which provides favorable conditions for polymerization of PAAc-NHS ester during the emulsion process. This prepared solution was added dropwise to 500 mL of corn oil with 0.1 wt % Tween 80. After stirring at 3000 rpm at 55° C. for 20 min, the mixture was stirred in an ice-water bath for another 2 h. To obtain the SHIELD particles, the mixture was filtered and washed with acetone. SHIELD particles were dried in a chemical hood thoroughly and stored in a desiccator for further use.

Fluorescent labeling of SHIELD particles. Briefly, Cyanine7 NHS ester (Lumiprobe) was dissolved in acetone at the concentration of 0.1 mg/ml, and SHIELD (1 g) was added to 10 ml dye acetone solution. The solution was stirred at room temperature overnight and SHIELD was collected through centrifugation. The collected SHIELD was further washed three times with acetone and dried in a hood before use.

Fourier-transform infrared spectroscopy and ¹H nuclear magnetic resonance (NMR) spectrum. The interaction between SHIELD and amino groups was revealed by Fourier-transform infrared spectroscopy (FT-IR) spectrum (Alpha, Bruker, USA). ¹H nuclear magnetic resonance (NMR) spectrum was performed with a Bruker Avance NEO 600 MHz NMR, equipped with a RT BBO smart probe, TXI 1H-13C/15N-2H probe, VT, and SampleXpress automatic sample changer.

Adsorption of mucin to microspheres. 5 ml of Mucin solution (0.5 mg/ml) was prepared, and SHIELD particles (0.5 mg) were added. After vortexing and incubation at 37° C. for 2 h, the mixture was centrifuged at 1375 g for 5 min. To determine the concentration of mucin, periodic acid reagent was added to the supernatant (1:10). After incubation at 37° C. for 2 h, Schiff reagent was added in the same volume as periodic acid. The optical density was measured at 555 nm using a microplate reader 30 min later. The concentration of mucin was determined using a calibration curve.

SEM imaging of SHIELD particles. Conductive tapes were placed on specimen stubs. SHIELD powder was dispersed on conductive tape to form a loose layer, and unattached powder was blown away using a bulb syringe. Samples were sputter coated with 10 nm Au plasma (30 s) before imaging. SEM images were taken using a JEOL (JCM-7000) under acceleration voltage 15.0 and the HV vacuum mode.

Rheology test. A Molecular Compact Rheometer (MCR302, Anton Paar, Graz, AUS) equipped with a cone plate geometry of 50 mm diameter and 1 degree (CP50-1/TG, Anton Paar, Graz, AUS) was used. Truncation gap was set at 0.1 mm. Before the measurement, instrument inertia was checked and the system was calibrated as routine. 2 mL of sample was placed on the temperature-controlled Peltier plate at RT. Geometry cone was set at the measuring position and excessed sample was trimmed. The following tests were performed in triplicate: Strain sweeps were performed at an oscillatory frequency of 1 Hz, Shear strain (y) ranging from 0.1%-100%, 60 data points were collected at log ramp of shear strain. Frequency sweeps were performed between 0.1 to 50 Hz at 1% shear strain. 30 data points were collected for each measurement.

Particle tracking. Confocal laser scanning microscopy (FluoView, Olympus, JPN) was used for particle tracking under 3D visualization mode. A pt-slide well (#81506, ibidi, USA) was filled with twenty microliters freshly collected porcine stomach mucus. 5 μL of latex beads (L9904, 0.1 μm mean particle size, Sigma, USA) were then loaded gently on top. The deposition of latex beads was continuously monitored for 20 min.

For the Brownian motion analysis, 500 μl mucus or mucus+SHIELD (1 mg/ml) was added in 12 well plates and gently stirred before incubated for 1 h. Latex beads were diluted to 100× and 10 μl of diluted beads were added to each well without mixing. The motion of beads was observed and recorded for 20 s using a fluorescence microscope (Revolve, ECHO). Single particle tracking was performed with ImageJ plugin (TrackMate). Mean square displacement (MSD) was quantified using MATLAB with a per-value class (@msdanalyzer).

Cell culture. Human Bronchial Epithelial Cells (HBEpC) were purchased from ATCC (PCS-300-010) and cultured in pre-coated flasks with complete human epithelial cell medium/w kit (H6621, Cell Biologics, USA). 3D airway model (502-3D-24, Cell Applications) was cultured according to the manufacturer's instructions and sliced (5 μm) before staining.

Mouse studies. All studies and protocols were approved by the Institutional Animal Care and Use Committee of North Carolina State University (protocol number 19-806-B). Male CD1 mice (aged 7 weeks) were obtained from Charles River Laboratory (Massachusetts, USA). Cy7-labeled SHIELD particles (3 mg per kg of body weight) were delivered to the CD1 mice via inhalation treatment using a customized dry powder inhaler (DPI). Biodistribution of SHIELD in mice was studied first. Mice were euthanized at 4, 8, 24, 28, 32 and 48 hours. All major organs were collected and imaged by IVIS. Then, the organs were cryo-sectioned for histology and immunofluorescence analysis. Mice inhaled SHIELD 4 h, 8 h, or 24 h before a challenge with SARS-CoV-2 pseudovirus with D614G mutated spike protein (C1120G, Montana Molecular) or SARS-CoV-2 pseudovirus with part of B.1.1.7 spike protein (D614G, E484K, N501Y, and K417N mutations) (C1122G, Montana Molecular). The dose of pseudovirus was 100 μl per mouse. GFP labelled Mouse Pneumonia Virus (20 μl per mouse, OTV-011, Creative Biogene) or H1N1 virus (100 μl per mouse, gamma radiation inactivated A/New Caledonia/20/99 strain, labeled with DiD, 23-047-299, Microbiologics). One day after challenge, all major organs were collected and evaluated by IVIS imaging. After that, organs were kept in 10% NBF followed by dehydration in 10%, 20% and 30% sucrose. 5 μm lung sections were prepared.

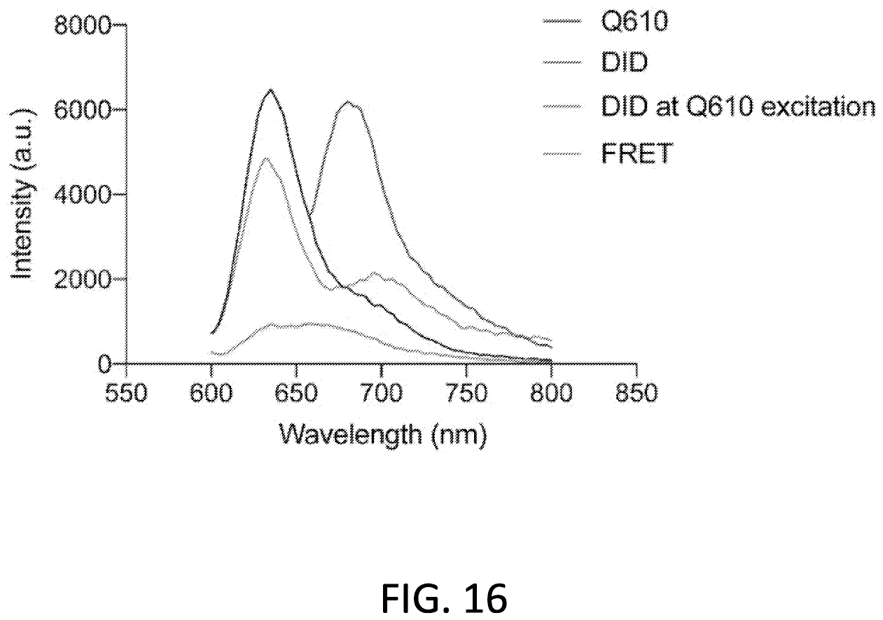

Characterization of H1N1 virus labelled with DiD. Förster resonant energy transfer (FRET) assay was applied using Q610 (donor, excitation wavelength 570 nm) to label anti-HA antibody (Influenza A H1N1 (A/New Caledonia/20/1999) Hemagglutinin/HA Antibody, Rabbit PAb, 1:200, 11683-RP01, SinoBiological) on the virus surface and DiD (acceptor, excitation wavelength 630 nm) as a FRET pair. FRET occurs when two molecules are close enough, where the emission of the donor can be partly utilized as the excitation for the acceptor.¹⁻³ The fluorescence emission of DiD was observed when virus particles were co-labelled with Q610 and DiD, while there was no fluorescence signal observed when DiD was excited at 570 nm (FIG. 16), indicating that the DiD is on the surface of the virus particles close to HA antigen.

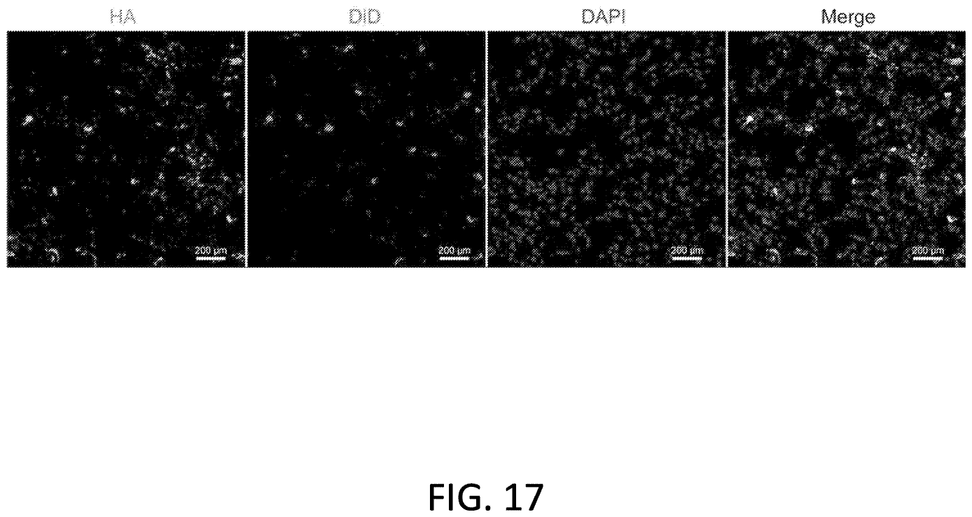

To further identify the fluorescence labelled virus particles, mouse lungs were harvested after challenging with DiD-labeled virus particles and stained with an antibody specific to H1N1 HA (FIG. 17). The colocalization of DiD and viral antigen was observed using confocal microscopy, revealing DiD labelled virus particles entering into cells.

Pulmonary function and sIgA measurements. Pulmonary function measurements were performed with the FlexiVent (SCIREQ Inc., Montreal, Canada). Prior to measurements, animals were anesthetized with an intraperitoneal injection of ketamine and xylazine solution (2:1 ratio). The animals were intubated with a cannula. Pulmonary function baseline data was recorded before inhalation on day 0. Mice inhaled SHIELD (3 mg/kg weight) daily for two weeks. For BAL fluid collection, a catheter was inserted in the trachea of the anesthetized mouse. 1 ml saline solution was instilled into the bronchioles through the catheter and retracted several times. The sIgA level was evaluated with an ELISA kit (ab157717, abeam) according to the instructions.

Pulmonary mucociliary clearance test in vivo. Mucociliary clearance (MCC) was determined by the elimination of microspheres from the lungs and nose. Mice were anesthetized and intratracheally instilled with 5×10⁶ carboxylate-modified yellow-green fluorescent microspheres (2 μm, F8827, Invitrogen). Mice were euthanized immediately (for the baseline measurement) or 45 min (for control and SHIELD groups) after treatment with microspheres. For the SHIELD group, mice were treated with SHIELD daily for two weeks or 2 h before the MCC test. Lungs with tracheas were harvested and placed in 0.1% Tween 20 PBS solution. Tissues were homogenized by a dissociator (gentleMACS) using a preset program. Homogenized tissue solution (10 μL) was read with an automated cell counter (Countess, ThermoFisher). The remaining fraction of microspheres was calculated by subtracting the quantity from baseline. Pulmonary mucociliary clearance was then calculated by subtracting the remaining fraction from 100%.

Histological analysis. For H&E staining, sections were fixed in formalin (Sigma-Aldrich) for 2 min and rinsed with running water. After staining in hematoxylin (Sigma-Aldrich) for 5 min, the sections were rinsed again. Afterwards, sections were dipped in pre-prepared acid alcohol for 2 s and rinsed with sodium bicarbonate. Sections were then stained with eosin (Sigma-Aldrich) for 2 min and washed with dehydrant until excess color was washed out.

Mucus collection and preparation. A stomach was harvested from eight weeks old male Yorkshire pig (ordered from Unit II Palmetto). The procedures complied with the ethical regulations approved by Institute Animal Care and Use Committee (IACUC) of North Carolina State University (protocol #: 20-137-B). The mucus was gently scraped off the stomach epithelium and diluted 1:5 in water supplemented with 200 mM NaCl, as well as 5 mM benzamidine HCl, 1 mM 2,4′-dibromoacetophenone, 1 mM phenylmethylsulfonyl fluoride, and 5 mM EDTA. The pH was adjusted to pH 7.4 with NaOH and the mixture was gently stirred overnight at 4° C. The solution was then centrifuged to remove cellular and food debris, and the supernatant was used for further study. For staining, mucus+SHIELD (2:1) was dissolved in PBS and gently spread on a slide. The slide was baked at 60° C. overnight to dehydrate liquid, fixed for 10 minutes in 3.7% buffered formalin solution, and then decontaminated using 70% ethanol and 1-5% phenol solutions. After washing in water, the slides were dried in air. Wheat Germ Agglutinin (Alexa Fluor 488 conjugated) was used for staining. After 3 h, slides were washed with PBS for further analysis.

SEM imaging of tracheas. Porcine tracheas were harvested and cut into pieces. SHIELD was spread evenly on the surface of tracheas. The swelling reaction was stopped by dipping the samples into 100% ethanol (30 s) for dehydration after 10 s or 10 min. Then, tissues were fixed in 2% buffered glutaraldehyde overnight, and then rinsed with 0.1 M HEPES buffer 3 times (5 minutes each). Series concentration of alcohol was used for dehydration as listed: 50% ethanol, 2 times for 10 minutes each with agitation. 70% ethanol, 2 times for 10 minutes each with agitation. 95% ethanol, 2 times for 10 minutes each with agitation. 100% ethanol, 3 times for 15 minutes each with agitation. Finally, tissues were dried through chemical drying: 100% EtOH:HMDS (2:1) for 15 minutes; 100% EtOH:HMDS (1:1) for 15 minutes; 100% EtOH:HMDS (1:2) for 15 minutes; HMDS alone for 15 minutes, 3 times. Samples stayed in a chemical hood overnight to evaporate the HMDS thoroughly. Samples were mounted on specimen stubs and sputter coated with 10 nm Au plasma before imaging. SEM images were taken using a JEOL (JCM-7000) under acceleration voltage 15.0 and HV vacuum mode.

IHC analysis in mouse studies. For immunofluorescence, lung cryosections were placed at room temperature for 30 min and then washed with PBS for 40 min. Primary antibodies were diluted with 0.01% saponin (Sigma-Aldrich) Dako solution. The antibodies were rabbit anti-firefly luciferase antibody (1:100; ab185924, Abcam), rabbit anti-Prosurfactant Protein C (proSPC) antibody (1:100; ab211326, Abcam), and mouse anti-SARS-CoV/SARS-CoV-2 spike antibody (1:100; 40150-D003, SinoBiological). All cryosections were incubated with primary antibodies overnight at 4° C. Secondary antibodies including goat anti-mouse IgG H&L (Alexa Fluor® 647, Abca) and goat anti-rabbit IgG H&L (Alexa Fluor® 488, Abcam) were diluted in a ratio of 1:200. After washing with PBS, samples treated with primary antibodies were incubated with those secondary antibodies at room temperature for 1.5 h. Afterwards, cryosections were mounted by using Prolong Gold Mounting Media with DAPI (Life Technologies). A confocal fluorescent microscope was used to image all the samples. For SARS-S immunohistochemistry staining, slides were incubated with primary mouse anti-SARS-CoV/SARS-CoV-2 spike antibody (1:100; 40150-D003, SinoBiological) overnight at 4° C. After washing with PBS, slides were incubated with goat anti-mouse HRP secondary antibody (1:200, ab97023, Abcam) for 30 minutes and then counterstained with hematoxylin for 1 min. DAB/AEC chromogen solution was added to cover the tissue sections for 1-10 min until colored precipitate appeared. All the images were analyzed using the NIH ImageJ software.

Nonhuman primate studies. 12 African Green Monkeys (AGMs) were allocated by a counterbalance randomization based on sex and weight. All animals were housed at Bioqual, Inc. (Rockville, MD, USA). SHIELD (3 mg/kg body weight) was administered by inhalation using a customized dry powder inhaler (DPI) and fitted mask 8 h before virus challenge. 6 AGMs were challenged with SARS-CoV-2 WA1 (SARS-CoV-2, isolate USA-WA1/2020, BEI-NR-53872, Lot #70040665) and 6 AGMs were challenged with SARS-CoV-2 B.1.617.2 (Delta) variant (SARS-CoV-2, isolate hCoV-19/USA/PHC658/2021, Delta Variant BEI-NR-55612, Lot #70045240) using the intranasal and intratracheal routes. The viral inoculum (0.5 mL) was administered dropwise into each nostril and 1.0 mL of viral inoculum was delivered intra-tracheally using a French rubber catheter/feeding tube, size 10, sterile (cut 4″-6″ in length). AGMs were inoculated with a total dose of 1.1×10⁵ PFU SARS-CoV-2. Bronchoalveolar lavage (BAL), nasal swabs (NS), blood, body weight, and body temperature were monitored or collected throughout the study. AGMs were necropsied on day 7 post-challenge. All immunologic and virologic assays were performed blinded. All animal studies were conducted in compliance with all relevant local, state, and federal regulations and were approved by the Bioqual Institutional Animal Care and Use Committee (IACUC).

Histopathology and immunohistochemistry in AGMs. After fixation in 4% paraformaldehyde for 24 hours, tissues were transferred to 70% ethanol and paraffin embedded. Tissue blocks were sectioned at 5 μm thickness. To rehydrate the tissue, slides were immersed in xylene 2 times for 10 minutes each. Afterwards, tissues were immersed in a series of graded ethanol including 100% (2 times, 10 minutes each), 95% (5 minutes), 70% (5 minutes) and 50% (5 minutes). After water rinse, slides were stained with hematoxylin (HSS16, Sigma-Aldrich) and eosin Y (318906, Sigma-Aldrich). An optical microscope was used for analysis. For SARS-N protein IHC staining, tissue sections were rehydrated as before and then treated with antigen retrieval buffer (AP9003125, Thermo) to enable antigen retrieval. Slides were incubated with primary rabbit anti-SARS-N antibody (NB100-56576, Novus, 1:200) overnight at 4° C. and then with goat anti-rabbit HRP secondary antibody (ab6721, Abcam, 1:1000) for 1.5 h. Finally, slides were counterstained with hematoxylin followed by bluing using 0.25% ammonia water.

Microscopic lung fibrosis was scored using the Ashcroft scale based on H&E staining, which utilizes a numerical scale from 0 through 8 to grade fibrosis. Quantification of RNAscope intensity was performed according to the manufacturer's instructions (https://acdbio.com/image-analysis), using ImageJ with Color Deconvolution and Weka Classifiers. For quantification of IHC, histochemical scoring (H-score) was performed to assess the interpretation of immunoreactivity. H-score incorporates both the staining intensity (i) and a percentage of stained cells at each intensity level (Pi), as previously described.⁴⁻⁷ The i values are indicated as 0 (no evidence of staining), 1 (weak staining), 2 (moderate staining), and 3 (strong staining). The Pi values vary from 0% to 100%. The final H-score is derived from the sum of i multiplied by Pi as in the equation shown below. This score, therefore, is in the range of 0 to 300.

$$H - score = ( 0 \times {P}_{0} ) + ( 1 \times {P}_{1} ) + ( 2 \times {P}_{2} ) + ( 3 \times {P}_{3} )$$

Subgenomic mRNA viral load assay. SARS-CoV-2 E gene subgenomic mRNA (sgRNA) was assessed by RT-PCR. SARS-CoV-2 E gene sgRNA was cloned into a pcDNA3.1 expression plasmid and transcribed to obtain RNA for standard curve generation (AmpliCap-Max T7 High Yield Message Maker Kit, Cellscript). The standard curve was used to calculate sgRNA in copies per mL or per swab. For RT-PCR, the collected samples were reverse-transcribed (Superscript III VILO, Invitrogen). A gene expression assay (Tagman, Thermo Fisher Scientific) was customized to target the E gene sgmRNA. The qPCR was performed on a QuantStudio 6 and 7 Flex Real-Time PCR System (Applied Biosystems) according to the manufacturer's specifications. The quantitative assay sensitivity was 50 copies per mL or per swab.

RNAscope in situ hybridization. Tissue slides were deparaffinized and rehydrated as previously described. The retrieval was performed according to the manufacturer's specifications. Briefly, slides were immersed in ACD P2 retrieval buffer (ACD Cat. No. 322000) at 95-98° C. for 15 mins and treated with protease plus (ACD Cat. No. 322331) at 40° C. for 30 min. SARS-CoV-2 anti-sense specific probe v-nCoV2019-S (ACD Cat. No. 848561) was used to target the positive-sense viral RNA. RNAscope® 2.5 HD Detection Reagents-RED (ACD Cat. No. 322360) was used for the probe hybridization and detection.

Immunofluorescence staining of AGM lung sections. All the slides were pre-treated as previously described, including rehydration and retrieval. Tissue slides were incubated with primary antibody (rabbit anti-SARS-N, 1:200) overnight at 4° C. The slides were then incubated with goat anti-rabbit Alexa Fluor® 594 (Abcam, ab150080, 1:500) and AF-488-CD206 (Santa Cruz Biotechnologies, sc-376108, 1:150) at RT for 1 hr. Olympus FLUOVIEW confocal microscope was used for imaging.

Statistics & Reproducibility. All experiments were performed at least three times independently. No statistical methods were used to pre-determine sample sizes but the sample sizes are based on previous studies. Animals were randomized to treatment groups. Data acquisition and analysis were performed by investigators blinded to the groups. No data was excluded from the analyses. Results are shown as means±standard deviation. Comparisons between two groups were performed using the two-tailed, unpaired Student's t-test or two tailed Mann-Whitney test for nonhuman primate study. Comparisons among more than two groups were performed using one-way ANOVA, followed by Tukey's test. p<0.05 was considered statistically significant.

### 4. EXAMPLES

The surge of fast-spreading SARS-CoV-2 mutated variants highlights the need for fast, broad-spectrum strategies to counteract viral infections. Embodiments of the present disclosure provide a physical barrier against SARS-CoV-2 infection based on an inhalable bioadhesive hydrogel, named spherical hydrogel inhalation for enhanced lung defense (SHIELD). Conveniently delivered via a dry powder inhaler, SHIELD particles form a dense hydrogel network that coats the airway, enhancing the diffusional barrier properties and restricting virus penetration. SHIELD protective effect is first demonstrated in mice against two SARS-CoV-2 pseudoviruses with different mutated spike proteins. Strikingly, in African green monkeys, a single SHIELD inhalation provides protection for up to 8 hours, efficiently reducing the infection against the SARS-CoV-2 WA1 and the B.1.617.2 (Delta) variants. Notably, SHIELD is made with food grade materials and does not affect normal respiratory functions. This approach could offer additional protection to the population against SARS-CoV-2 and other respiratory pathogens and insults.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

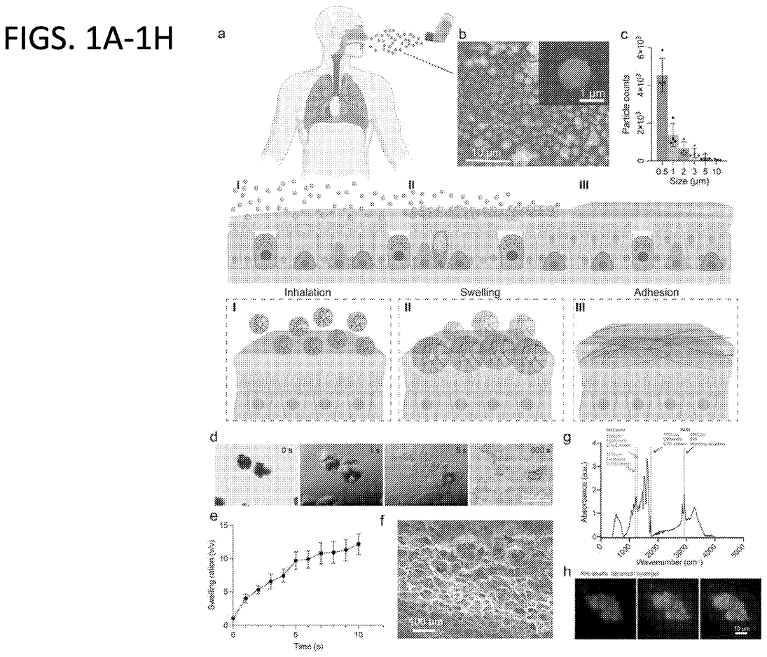

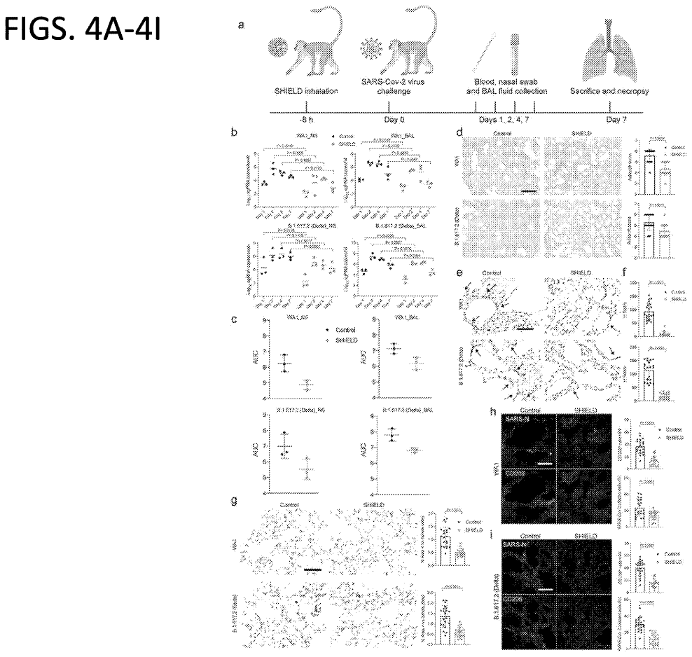

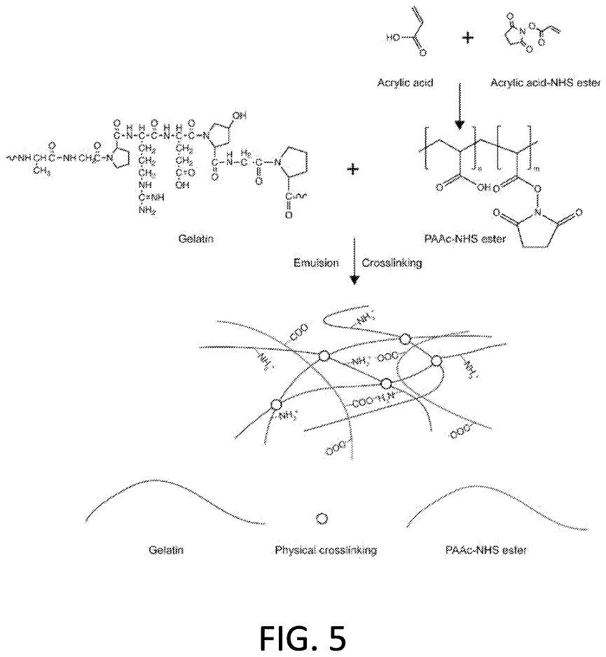

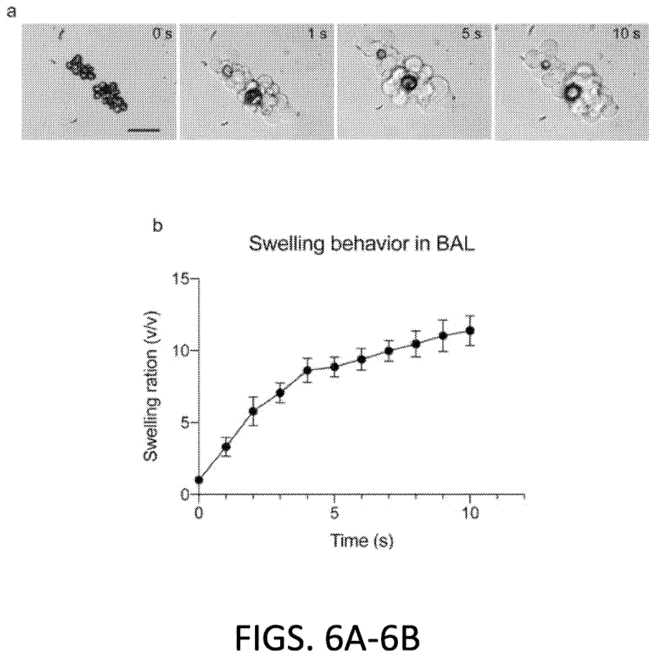

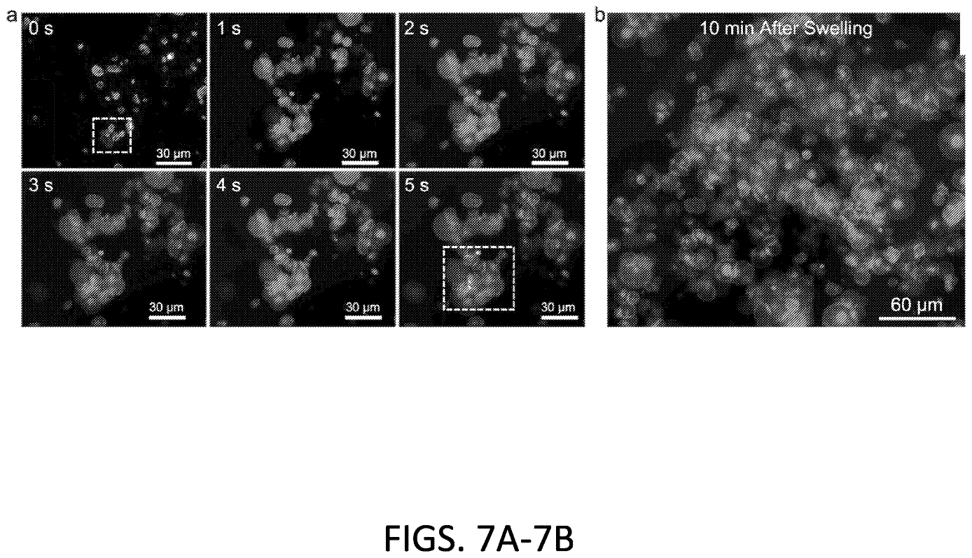

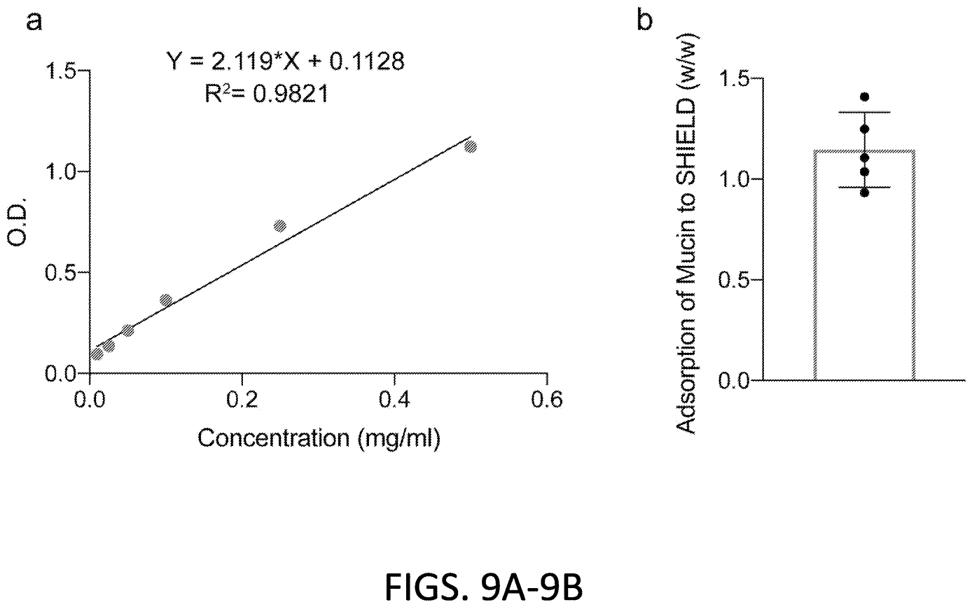

Fabrication and characterization of SHIELD particles. SHIELD takes effect in three steps (FIG. 1A), including Inhalation (I), Swelling (II) and Adhesion (III). Preparation of the SHIELD particles was carried out using the water-in-oil emulsion technique. The resultant microparticles are composed of crosslinked networks of poly(acrylic acid) grafted with N-hydroxysuccinimide ester (PAA-NHS ester) and gelatin (FIG. 5), which provides a mucoadhesive with robust mechanical properties. During the emulsion process, the physical crosslinking facilitates the transformation of the loose hydrogel structure to a dense spherical structure. SEM imaging revealed that SHIELD particles were well dispersed in powder status with a size below 5 μm (FIG. 1B). The aerodynamic diameter of SHIELD particles was in the range of 0.5-5 μm (FIG. 1C), favoring deep lung deposition through inertial impaction and sedimentation. SHIELD particles formed a hydrogel-like structure after swelling in water (FIG. 1D). The swelling kinetics were recorded as volume change (FIG. 1E). The volume of SHIELD increased >10 times within ten minutes. A similar swelling behavior was found in bronchoalveolar lavage (BAL) fluid (FIGS. 6A-6B). To enable tracking, SHIELD particles were labelled with Cyanine7. This labeling did not affect the swelling behavior (FIGS. 7A-7B). The stability of labelled SHIELD was evaluated. The fluorescence signal was stable during a 72 h period (FIG. 4). After swelling, SHIELD particles crosslinked with each other to form a hydrogel network structure (FIG. 1F). The FTIR spectrum displayed the presence of PAAc-NHS ester within SHIELD (FIG. 1G). The carboxylic acid C═O stretch at 1,700 cm⁻¹ and typical C—H stretching vibrations at 2940 cm⁻¹ is associated with PAAc in the SHIELD. The symmetric C—N—C stretch at 1,210 cm⁻¹ and asymmetric C—N—C stretch at 1,295 cm⁻¹ is associated with NHS ester in the SHIELD. The adsorption of mucin to SHIELD was quantified at a weight ratio of 1.14 (each mg of SHIELD binds 1.14 mg of mucins; FIG. 9). This result suggested that the mucoadhesion happens when SHIELD contacts with the wet surfaces of mucus. The hydration and swelling of SHIELD starts at the beginning of mucoadhesion, during which the negatively charged carboxylic acid groups in the PAAc-NHS ester can facilitate the process. Simultaneously, these carboxylic acid groups form intermolecular bonds (for example, hydrogen bonds and electrostatic interactions) with the tissue surfaces. Moreover, the NHS ester groups grafted on the PAAc can also covalently couple with the primary amine groups on tissues for further adhesion, which was confirmed by incubating SHIELD particles with amine-coupled fluorescent beads (FIG. 1H).

### Example 2

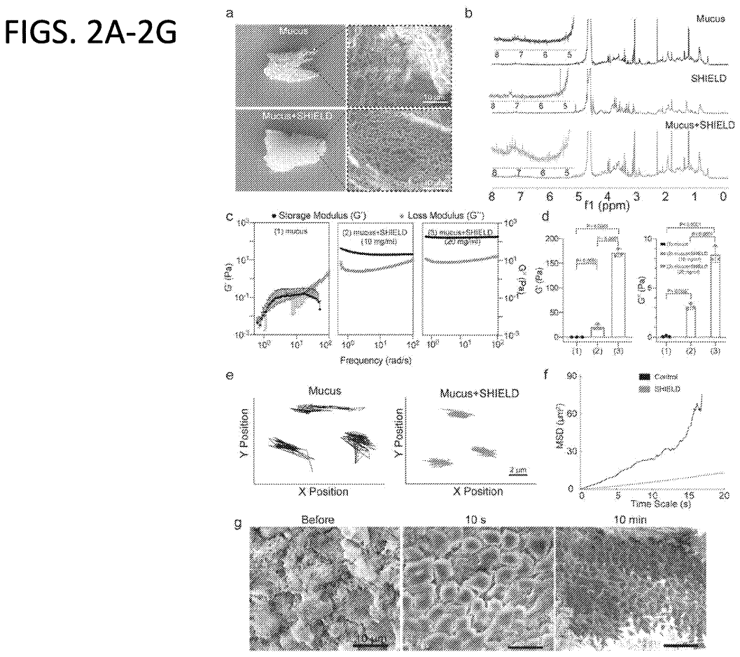

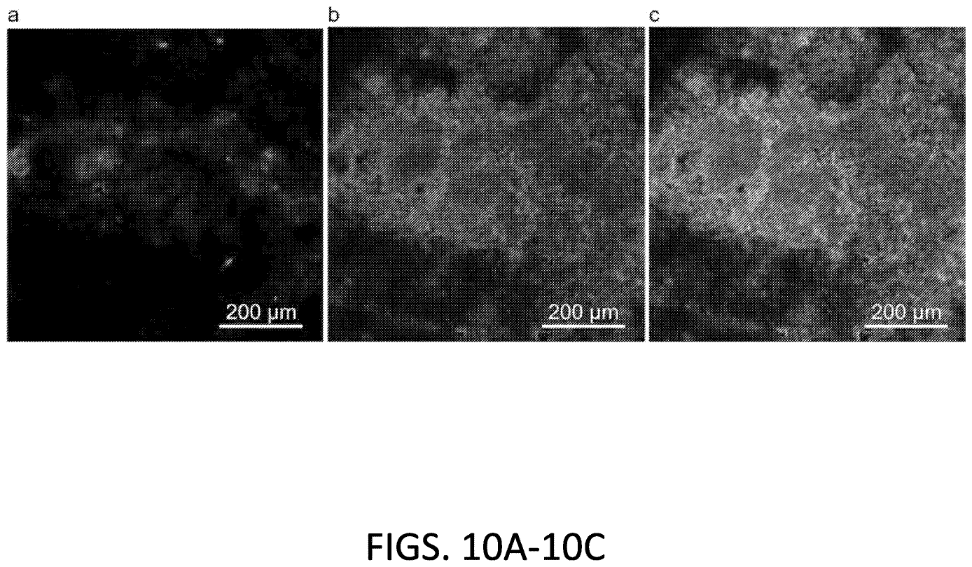

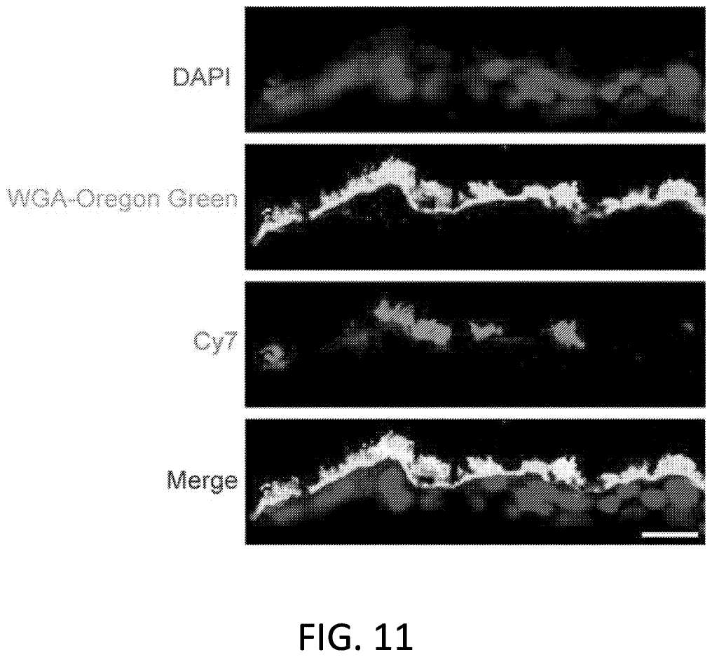

SHIELD hydrogel network reinforces the mucus barrier. It was hypothesized that the SHIELD could reinforce the mucus barrier by its swelling and adhesion behavior. Due to the limited volume of airway mucus in porcine tracheal tubes, porcine stomach mucus was collected for the test. Gastric mucins are encoded by MUC5AC and MUC6 genes, while airway mucins are encoded by MUC5AC and MUC5B genes. MUC5AC, MUC5B, and MUC6 share sequence similarities and common macromolecular characteristics, and all of them have regions rich in cysteine residues (Cys domains). Compared to mucus alone, mucus+SHIELD exhibited a denser morphology with smaller porous size (FIG. 2A), which may result from the interaction of SHIELD with mucus. The interaction of SHIELD and mucin was further observed through microscopic examination with wheat germ agglutinin staining (green) (FIG. 10) and in a 3D airway model (FIG. 11). ¹H NMR spectrum confirmed enhanced chemical shifts between 5.5 and 8 ppm (characteristic proton in amide —CONH group) in mucus+SHIELD (FIG. 2B), suggesting the interaction between the carboxyl groups (on SHIELD) and the primary amine groups (on mucus). With a higher intensity compared to SHIELD itself, more amide bonds could potentially form between SHIELD and mucus. Rheology studies (FIGS. 2C-2D) revealed that both the elastic (G′) and viscous (G″) moduli of mucus were significantly increased with SHIELD.

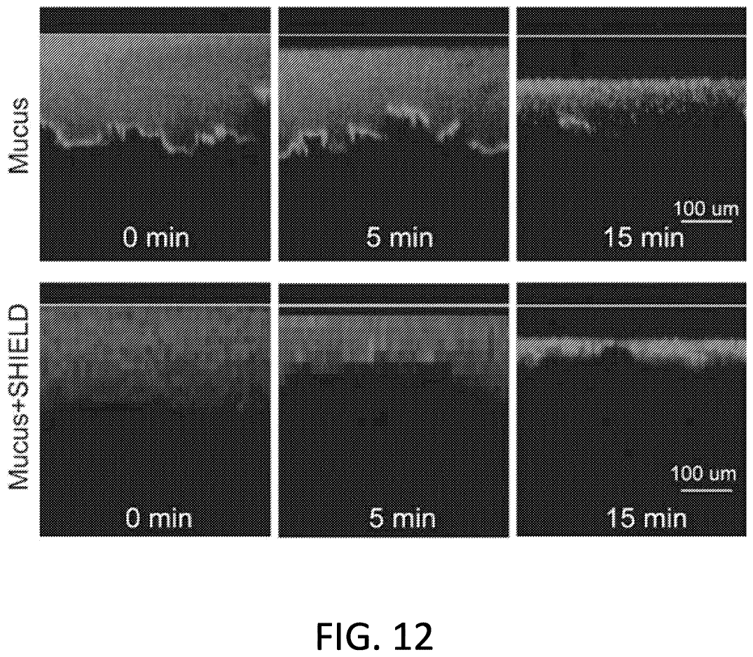

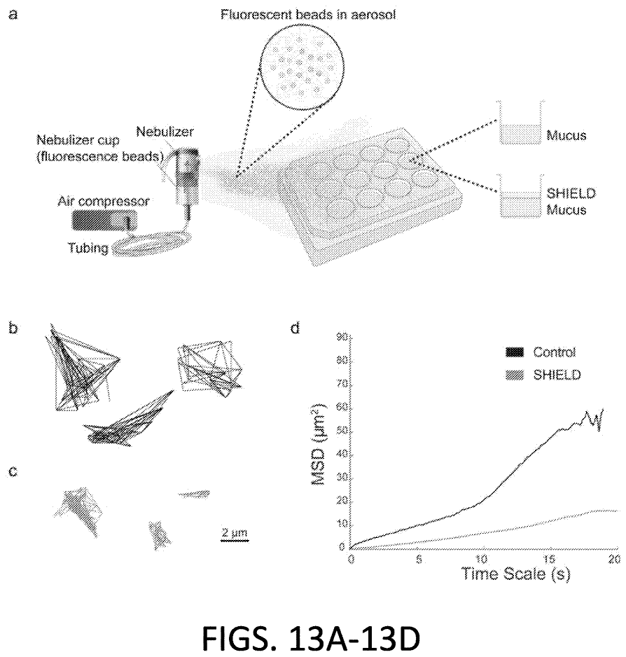

To simulate the penetration of virus through the mucus barrier, polystyrene particles of the same size as virus were used. By tracking the trajectories of particles, it was found that the addition of SHIELD in the mucus significantly reduced the Brownian motion of polystyrene particles (FIG. 2E). The restricted diffusion was evaluated via the quantification of the mean square displacement (MSD) (FIG. 2F). SHIELD reinforced the mucus barrier to decrease particle penetration (FIG. 12). Since the transmission of SARS-CoV-2 is by inhalation of respiratory aerosol, a nebulizer was used to generate aerosol containing polystyrene beads (FIG. 13A), which are similar in size to air-born viral particles. The trajectories of particles in control and SHIELD groups were analyzed (FIGS. 13B-13C). As shown in FIG. 13D, the motion of particles in the SHIELD group was restricted, with MSD reduced by 5-fold. In addition, the morphological change of the mucus layer with SHIELD was observed on the surface of porcine tracheal tube (FIG. 2G). Compared to the original mucus layer, interaction of SHIELD with mucin layer after spray result in a denser network morphology after 10 min.

### Example 3

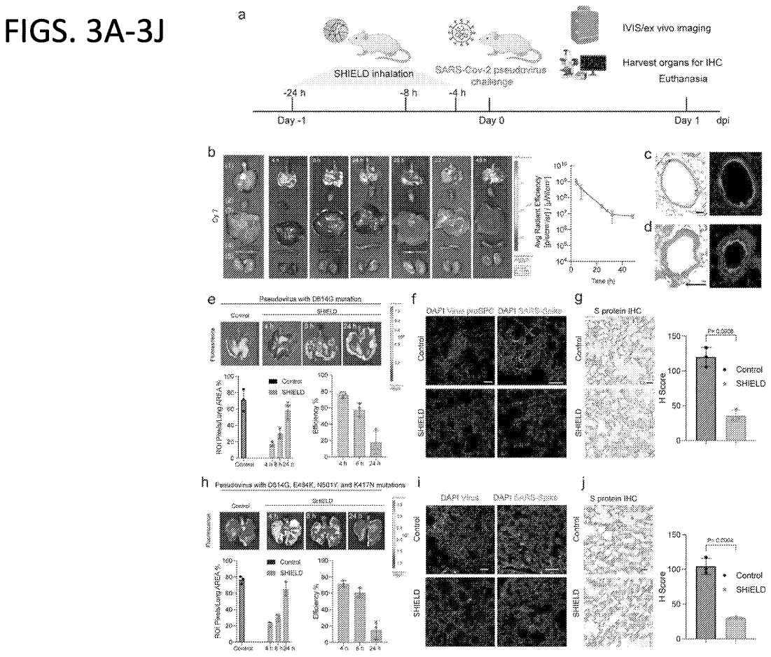

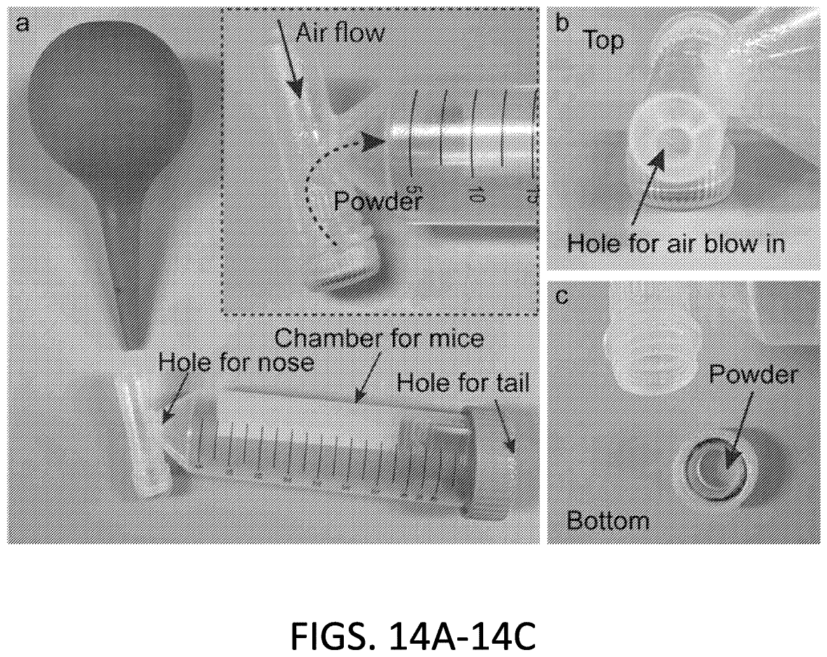

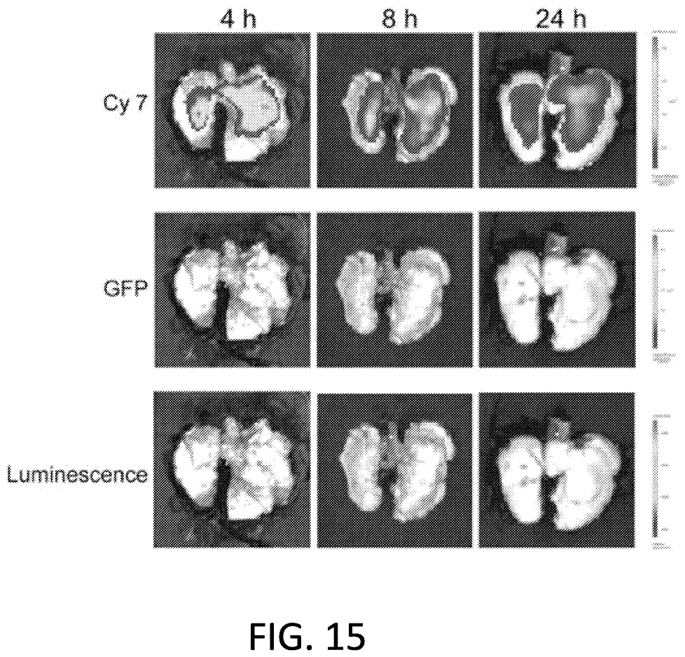

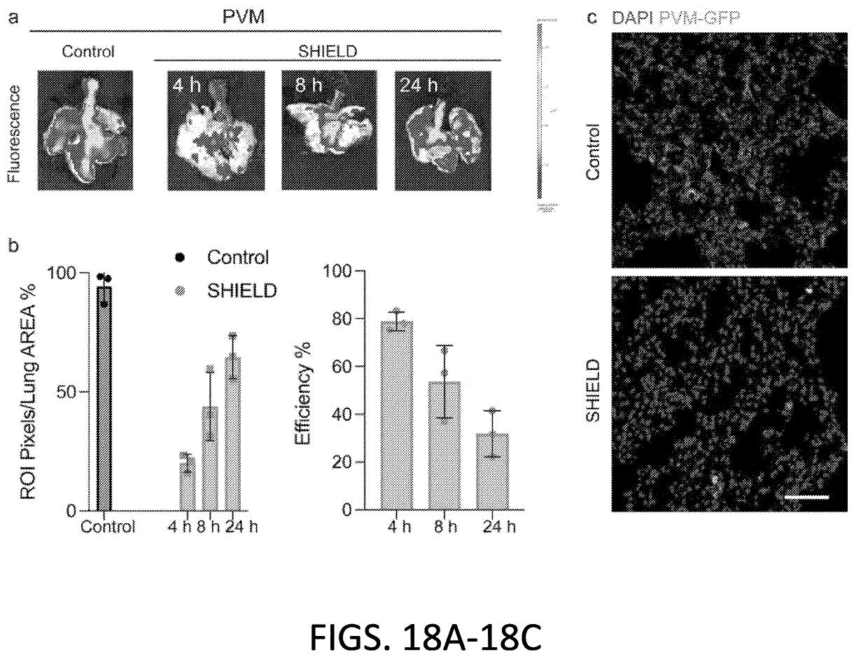

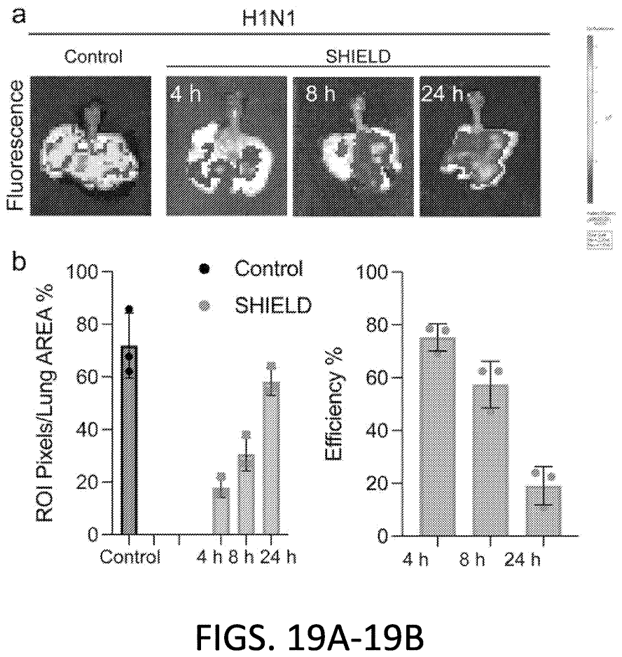

SHIELD inhalation blocks SARS-CoV-2 pseudo viruses in a mouse model. Before a live SARS-CoV-2 model, the protective effects of SHIELD were first tested in a mouse model of SARS-CoV-2 pseudovirus challenge (FIG. 3A). Inhalation in mice was performed with a modified “nose only” apparatus at a dose of 3 mg SHIELD per kg of body weight (FIG. 14). As shown in FIG. 3B, the signal of SHIELD remained high in the lungs 8 h after a single inhalation but decreased from 8 h to 24 h with minimal signal after 48 h, indicating the clearance of SHIELD. A control study showed that Cy7 labelled SHIELD did not interfere with the luminescent/fluorescent signal in IVIS (FIG. 15). As for the fluorescence signal which appeared in the liver, it was speculated that the clearance is also through liver metabolization after air-to-blood translocation. In addition, mucus turnover can contribute to the clearance of SHIELD through the mucociliary clearance mechanism. H&E staining and IHC indicated that inhaled SHIELD coated the surfaces of both bronchus (FIG. 3C) and small bronchioles (FIG. 3D). To study the protection of SHIELD towards variants, both SARS-CoV-2 pseudo type virus with D614G mutations and D614G, E484K, N501Y, and K417N mutations were intranasally instilled in mice 4 h, 8 h, or 24 h after SHIELD inhalation. IVIS imaging revealed that pre-inhalation with SHIELD blocked viral retention (baculovirus pseudotyped with D614G mutations) in the lung, with a blocking efficiency of 75.8% at 4 h, 57.7% at 8 h, and 17.7% at 24 h (FIG. 3E). Confocal microscopy (FIG. 3F) and S protein IHC (FIG. 3G) confirmed that inhalation of SHIELD reduced the amount of SARS-CoV-2 pseudovirus in the lung tissue. Similar protection efficiency was observed in mice challenged with baculovirus pseudotyped with part of the B.1.1.7 spike protein (D614G, E484K, N501Y, and K417N mutations), showing a blocking efficiency of 71.8% at 4 h, 60.6% at 8 h, and 15.1% at 24 h (FIG. 3H). This was confirmed by immunofluorescence staining (FIG. 3I) and S protein IHC (FIG. 3J). To study the broad-spectrum protection effects of SHIELD, those particles were tested against mouse pneumonia virus (PVM) and H1N1 flu virus in mice. The H1N1 virus was labelled with DiD before challenging, which was confirmed by Förster resonant energy transfer (FRET) assay and the co-staining of lung sections (FIGS. 16 and 17). These results indicated that SHIELD protection was also effective for those two pathogens (FIGS. 18 and 19).

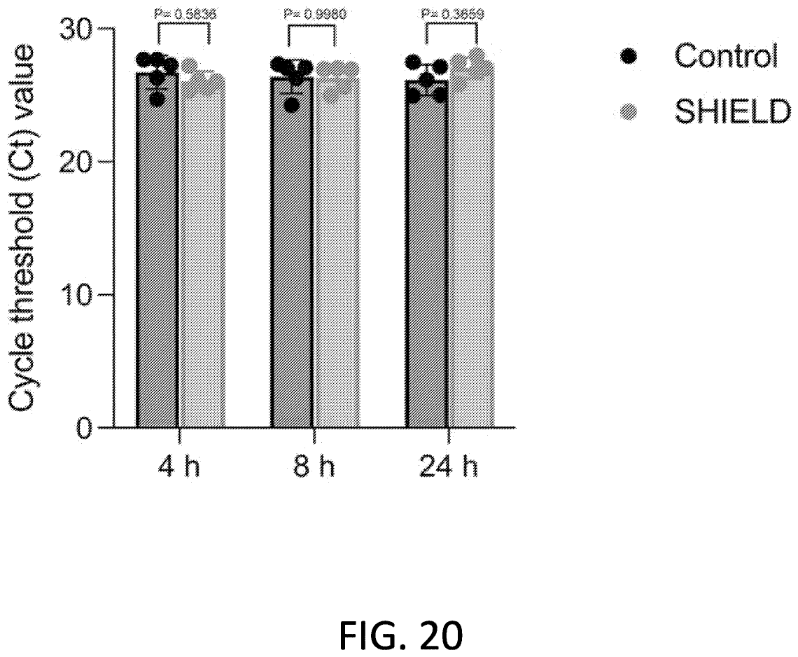

The protection of SHIELD in post-infection situations was also investigated using PVM, where mice were challenged before SHIELD inhalation. Virus shedding from the upper respiratory tract, which is regarded as a marker of infectiousness to predict the efficiency of viral transmission, were collected by nasal swab at different time points after SHIELD inhalation (4 h, 8 h, and 24 h). There was no difference between the control group and the SHIELD inhalation group (FIG. 20) in the quantification through PCR, indicating the minimal efficacy of SHIELD on post-infection individuals.

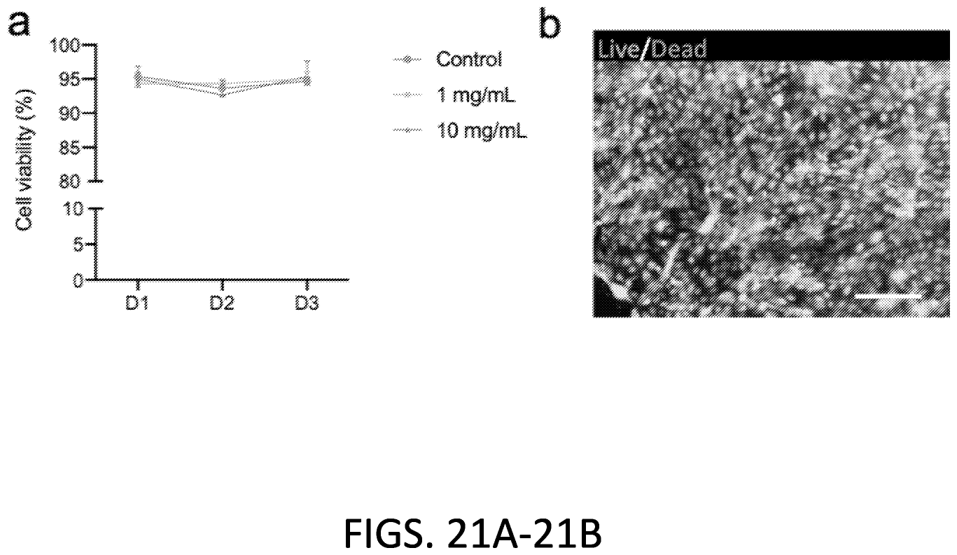

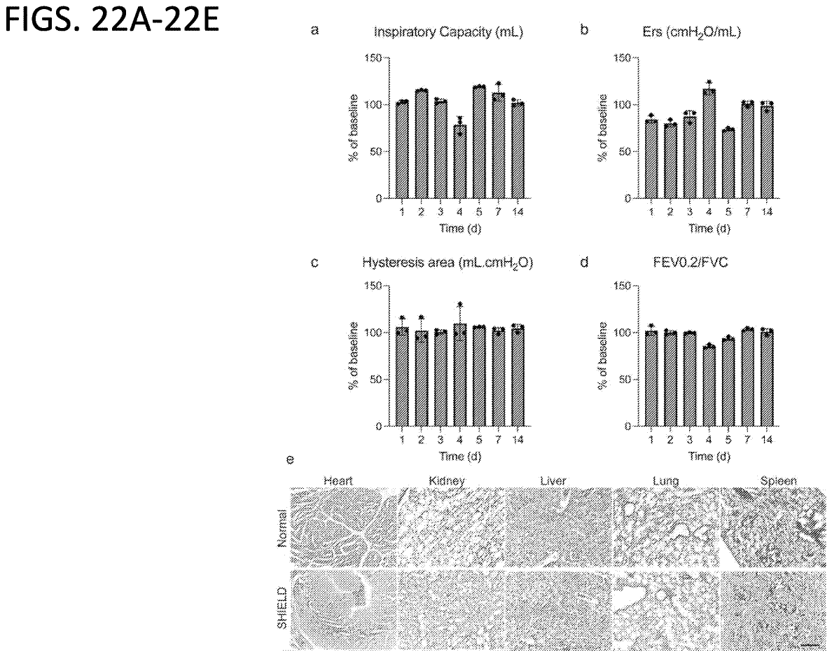

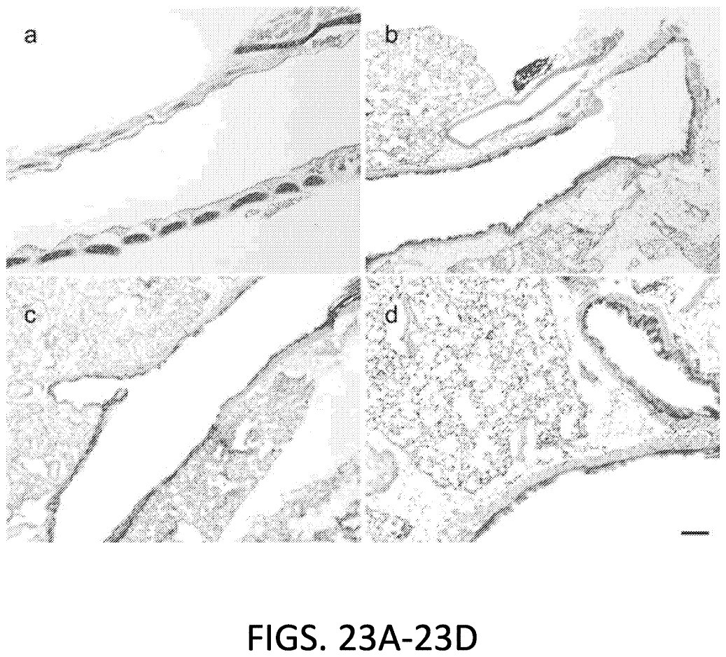

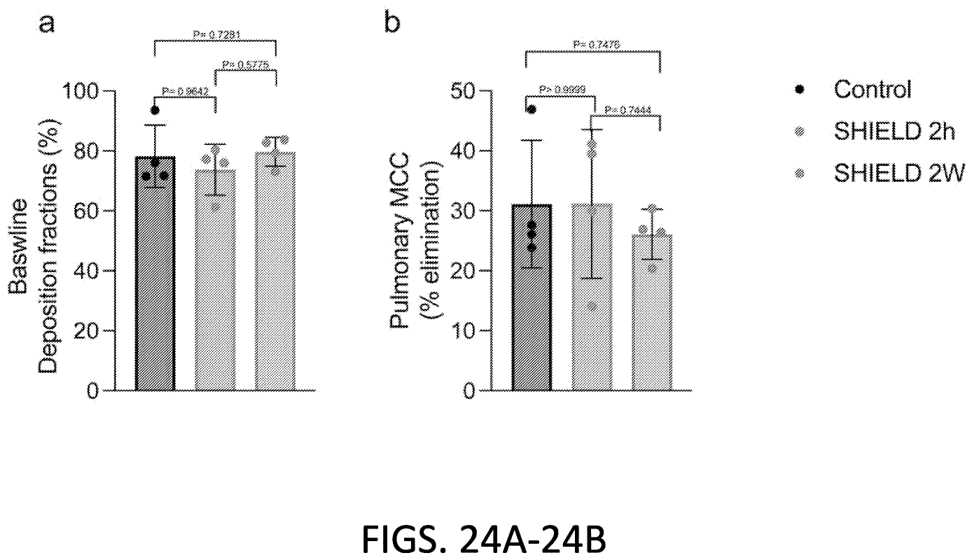

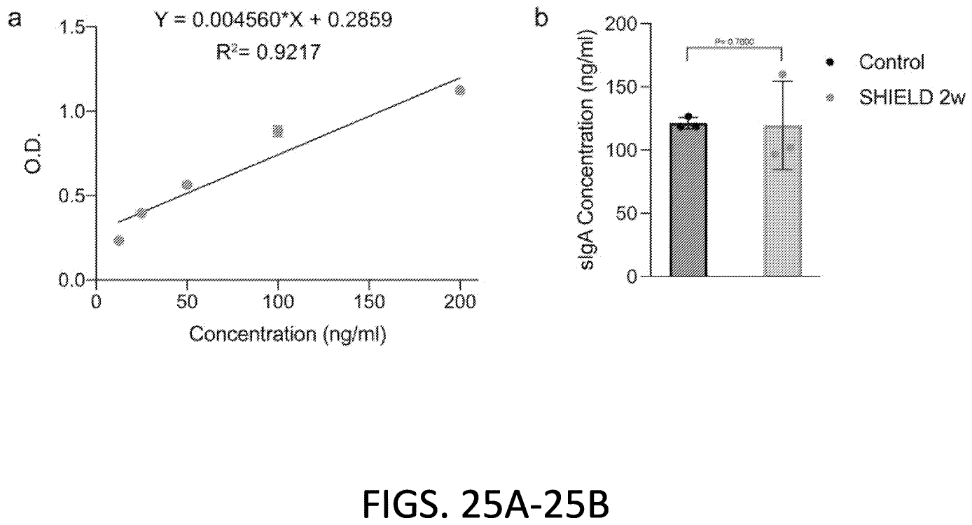

To study the biocompatibility of SHIELD, cells were cultured with SHIELD at different concentrations for three days. Even at high concentration (10 mg/ml), cell viability maintained at 95% (FIGS. 21A-21B). To study the toxicity of repeated SHIELD dosing, mice were treated with SHIELD daily for two weeks. All pulmonary function tests (PFTs) returned with a normal reading (80%-120% of baseline), including inspiratory capacity (IC), respiratory elastance (Ers), hysteresis area, and ratio of forced expiratory volume to forced vital capacity (FEV/FVC) (FIGS. 22A-22D). This indicated that the inhalation of SHIELD did not affect normal lung functions. After two weeks of dosing, major organs were harvested for H&E staining. There was no histological evidence of injury observed compared to control animals (FIG. 22E). Tracheas and bronchi harvested from these mice had normal morphologies (FIG. 23), suggesting the safety of repeated administration of SHIELD. To determine if the inhalation of SHIELD affects the normal function of mucociliary clearance (MCC), mice were treated with SHIELD either acutely (2 h) or long term (daily inhalation for 2 weeks). The results suggested that inhalation of SHIELD had minimal effects on pulmonary MCC (FIG. 24), and did not cause long-term crosslinking of mucus. Like other reported biopolymers, SHIELD particles only induced a temporary modification of mucus. Since the mucus layer acts not only as a physical barrier but also a biochemical barrier, the level of secretory immunoglobulin A (sIgA) antibody in the BAL fluid was evaluated before and after interaction with SHIELD. ELISA indicated unchanged sIgA levels after daily SHIELD inhalation for two weeks (FIG. 25), suggesting that mucosal immunity was not harmed by SHIELD administration.

### Example 4

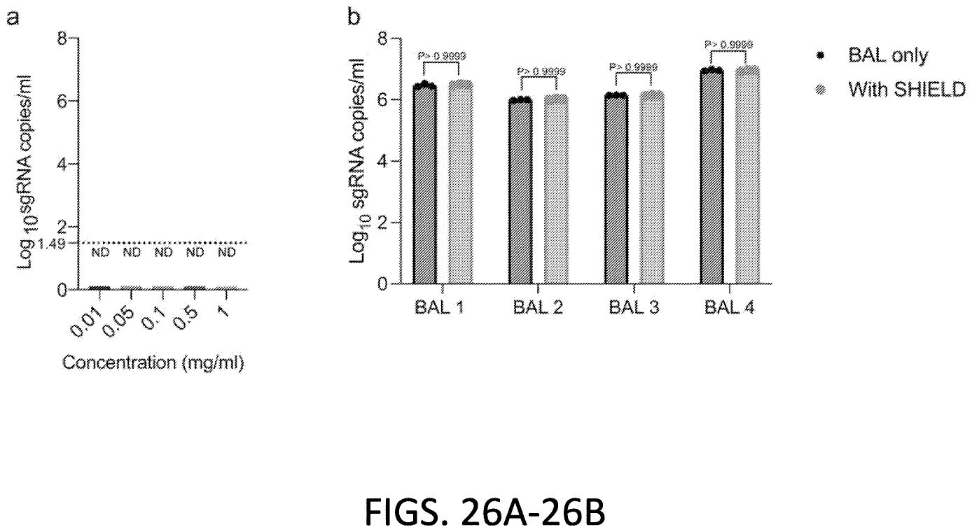

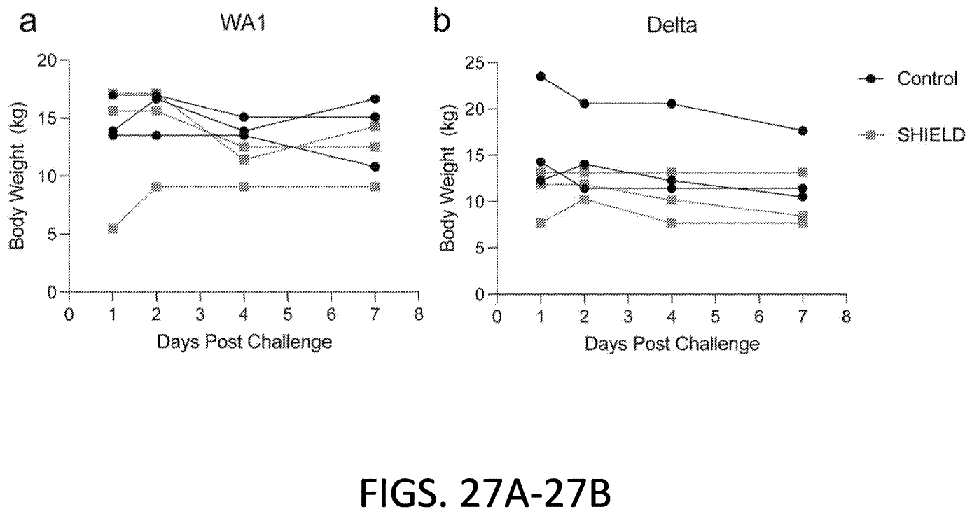

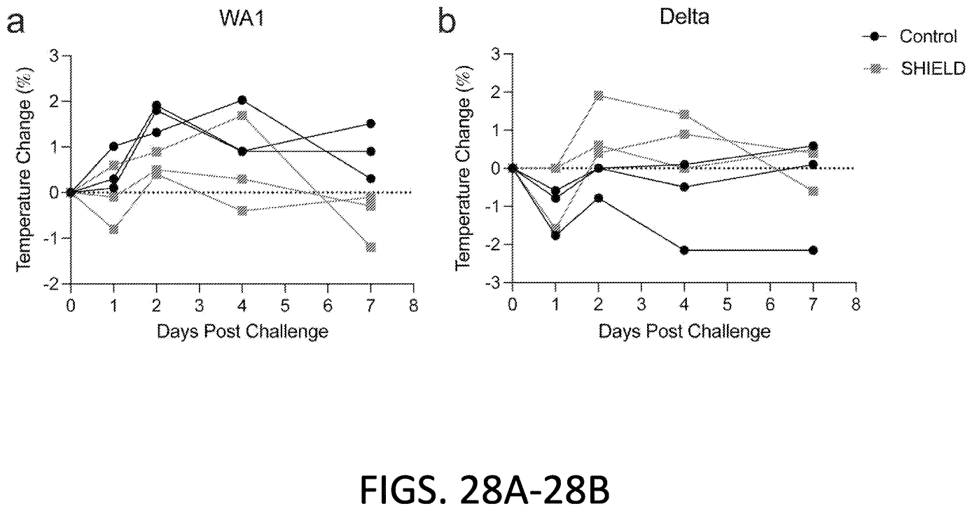

SHIELD inhalation protects African green monkeys from SARS-CoV-2 infection. To evaluate the protection efficacy of SHIELD, a pilot non-human primate study was performed using both the original SARS-CoV-2 WA1 and the B.1.617.2 (Delta) variant. SHIELD inhalation was performed 8 h before virus challenge, using either SARS-CoV-2 WA1 or the B.1.617.2 (Delta) variant by intranasal and intratracheal routes (FIG. 4A). Nasal swabs (NSs) and bronchoalveolar lavage (BAL) were collected on days 1, 2, 4, and 7. Viral load assay was performed by real-time PCR with reverse transcription specific for viral subgenomic RNA (sgRNA, indicative of virus replication) (FIG. 4B). Overall, animals protected with SHIELD had viral loads 50-300-fold less than the control animals (FIG. 4C). There was no effect of SHIELD itself on the PCR results (FIG. 26). The protection was seen with both SARS-CoV-2 WA1 and the B.1.617.2 (Delta) variant. Temperature and body weight fluctuations were not remarkable in both control and SHIELD-protected animals (FIGS. 27 and 28). According to the literature, transient fever may be a feature of disease in NHP models of COVID-19, but temperature changes were not consistently observed in many cases. Viral load is the direct evidence to see the virus infection. Although NHP are not able to develop all aspects of overt debilitating COVID-19 clinical illness as seen in humans, such as respiratory symptoms, they are still considered a gold standard model for developing new vaccines and therapeutics.

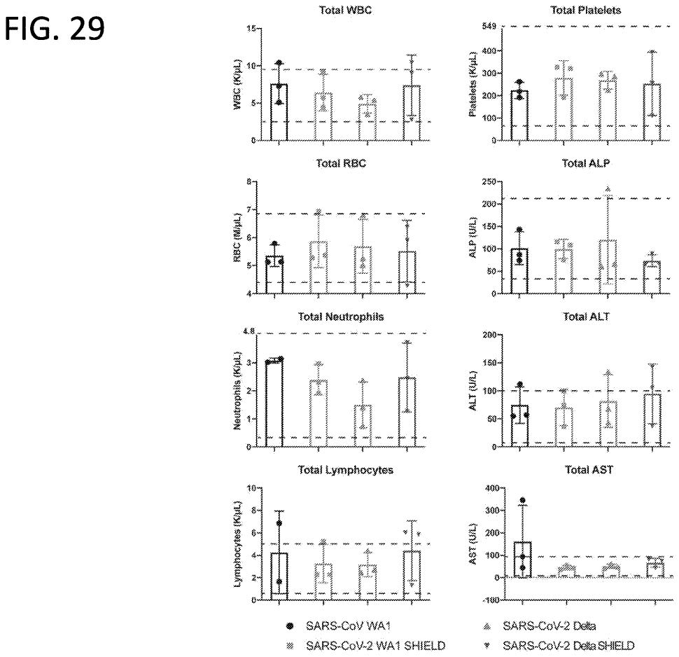

Hematoxylin and eosin (H&E) staining revealed more severe inflammation and viral pneumonia in the control animals (FIG. 4D). For the NHP model, the formation of fibrosis can be observed 5 days post infection, and the pulmonary pathological change of fibrosis is a marker for lung injury by interstitial pneumonia. Ashcroft score was applied here to measure the fibrosis, which revealed that SHIELD protection significantly decreased lung fibrosis. In addition, SARS nucleocapsid protein (SARS-N) was decreased substantially by SHIELD protection (FIGS. 4E-4F). To visualize SARS-CoV-2 viral RNA (vRNA) in the lung tissue, in situ RNA hybridization (RNAscope) was performed (FIG. 4G). Positive-sense vRNA was largely reduced in the SHIELD group compared to the control group, indicating limited viral replication. Furthermore, less SARS-N protein and fewer CD206⁺ macrophages were observed in the SHIELD group (FIGS. 4H-4I). As a macrophage marker, CD206 has been suggested to inhibit effector T cell function in SARS-CoV-2. The decrease of CD206⁺ macrophages with SHIELD protection indicated a reduced virus infection. Lastly, hematology analysis assured that SHIELD inhalation did not cause toxicity in the animals (FIG. 29).

### Example 5

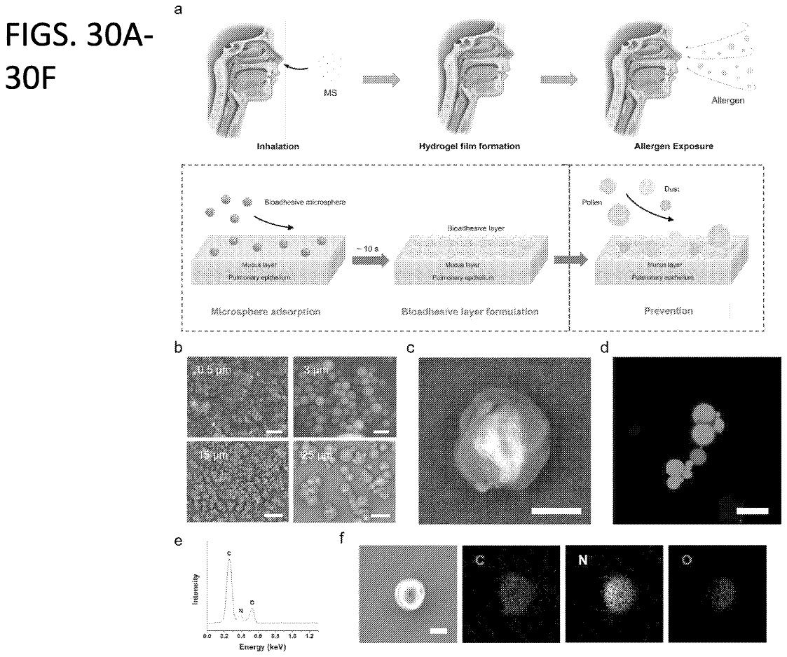

FIGS. 30A-30F provide experimental results involving the fabrication and characterization of bioadhesive microspheres. FIG. 30A provides a schematic representation showing airway protection by inhalable bioadhesive microsphere. In summary, dry bioadhesive microsphere powder is sprayed and inhaled through the nasal cavity, promptly adhering to the airway surface. Upon contact with mucus, they begin to swell and form a hydrogel that firmly attaches to the epithelium. Subsequently, inhaled airborne particles such as pollen and dust are captured and absorbed by the bioadhesive hydrogel, preventing potential harm to the body. Different-sized microspheres can be synthesized by adjusting the preparation conditions, ranging from 0.5 μm to 25 μm (FIG. 30B). The microspheres are uniform and exhibit a spherical structure (FIGS. 30C-30D). Elemental analysis (FIG. 30E) and mapping images (FIG. 30F) reveal the uniformly distributed C and O in the microspheres, originating from acrylic acid and gelatin. The N primarily arises from the amino group of protein in gelatin.

### Example 6

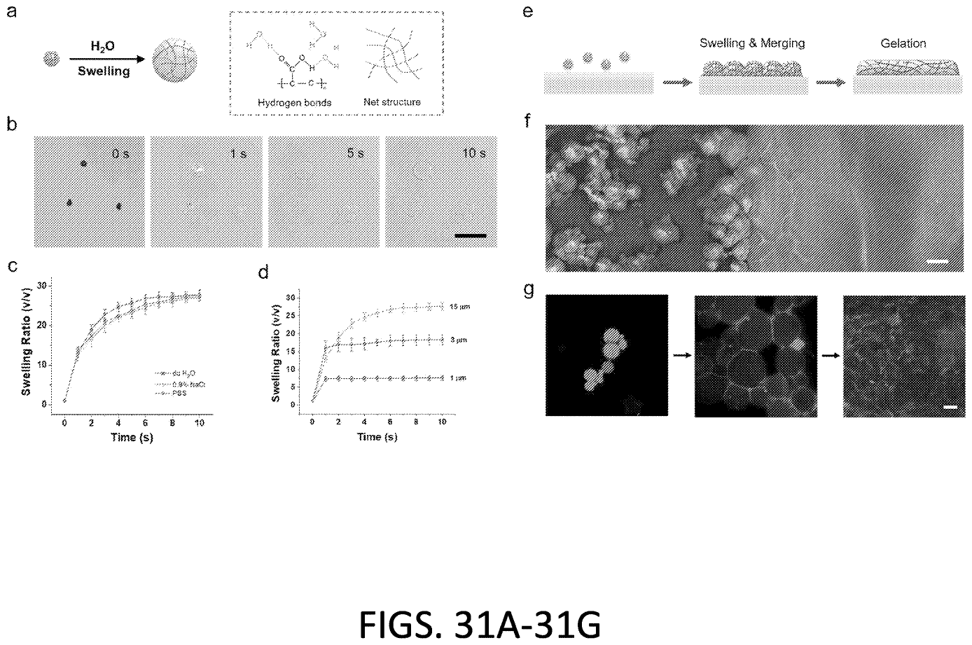

FIGS. 31A-31G provide experimental results pertaining to the swelling behavior of bioadhesive microsphere and hydrogel formation. The bioadhesive microsphere contains carboxyl groups in polyacrylic acid, which readily form hydrogen bonds with water. Its net structure has ample space to accommodate water molecules (FIG. 31A). Dry bioadhesive microspheres rapidly swell, reaching their maximum volume in less than 10 seconds upon contact with water (FIG. 31B). Their volume expands nearly 30 times in water, PBS, and normal saline (FIG. 31C). The swelling ratio is associated with their size, with larger microspheres exhibiting greater water-absorbing capacity (FIG. 31D). The gelation process was characterized by SEM (FIG. 31F) and fluorescent imaging (FIG. 31G). These images shows dry microspheres are separated and spherical. Upon contact with water, they start to swell, merge, and eventually form a complete hydrogel.

### Example 7

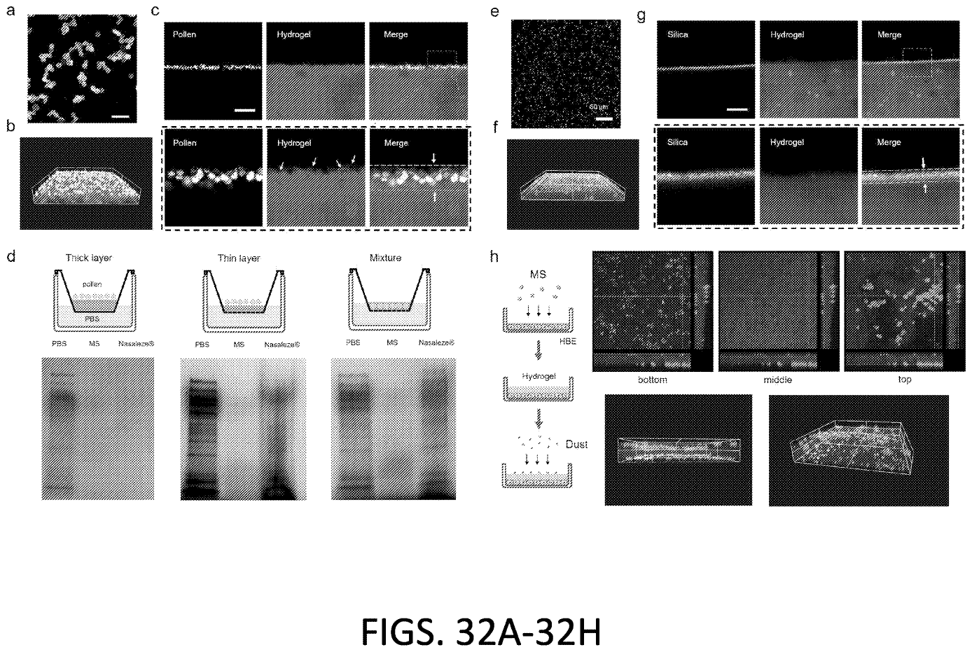

Experiments were conducted to assess the allergen adsorption capability of the bioadhesive hydrogel using a confocal fluorescent microscope (FIGS. 32A-32H). For visualizing pollen adsorption on the hydrogel, Rhodamine B-labeled pollen particles (FIG. 32A) and cy5-labeled bioadhesive microspheres were employed. The dry microspheres were sprayed onto the water layer to form a hydrogel, followed by pollen spraying. In the 3D confocal fluorescent imaging, pollen particles were observed to be absorbed and deposited onto the hydrogel surface (FIG. 32B). Enlarged images provided a detailed view of the adsorption interface (FIG. 32C): pollen particles were mixed with the hydrogel in a confined area, with a depth approximately the size of a few pollen particles. The cy5 channel illustrated that pollen particles were wrapped and coated with hydrogel, indicating the “soft surface” of the bioadhesive hydrogel.

To evaluate the allergen isolation ability of the bioadhesive hydrogel, a comparison was made with a commercial nasal spray protector. Results indicated that both bioadhesive microspheres and Nasaleze® could prevent the permeation of proteins released from pollen particles when the formed hydrogel layer was thick (~2 mm). However, as the thickness of the hydrogel decreased or when the hydrogel was directly mixed with pollen particles, the commercial product was unable to prevent the permeation of allergen proteins. In contrast, the bioadhesive hydrogel retained its ability to hold allergens, attributed to the chemical binding between NHS groups in the hydrogel and amino groups in allergen proteins.

The adsorption of silica on the hydrogel was also characterized using a confocal fluorescent microscope. Silica particles, labeled with Rhodamine B (FIG. 32E), were observed to deposit on the hydrogel surface after spraying (FIG. 32F). The interface between silica and hydrogel was observed (FIG. 32G), and it appeared thinner than the pollen-hydrogel interface due to the smaller size of the silica particles (1~5 μm). To assess the dust isolation capability of the hydrogel from cells, dry microspheres were sprayed onto human bronchial epithelial cells to form a hydrogel layer, followed by the spraying of real dust. 3D fluorescent images revealed that the bioadhesive hydrogel effectively isolated dust from the cells, preventing their direct contact (FIG. 32H).

### Example 8

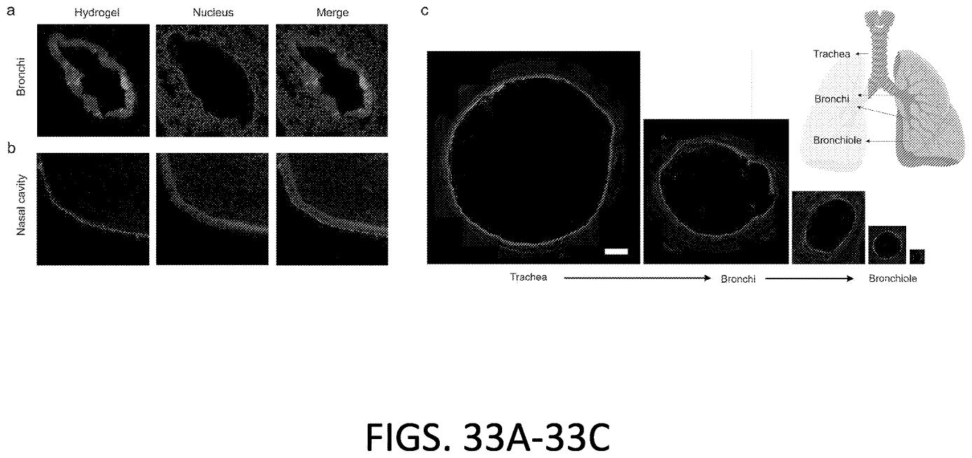

The in vivo formation of the protective hydrogel compositions on airways was assessed in both mice and pigs (FIGS. 33A-33C). Two different sizes of microspheres were employed for the protection of the trachea/bronchus (3 μm microsphere) or nasal cavity (15 μm microsphere). The figures illustrate that both the trachea and bronchus were effectively covered with a hydrogel layer formed by 3 μm microspheres (FIG. 33A). In the case of the 15 μm microspheres, they accumulated in the nasal cavity, forming a hydrogel film on the nasal mucosa (FIG. 33B). Subsequently, the formation and deposition of the bioadhesive hydrogel on airways were measured in porcine lungs. The airways, spanning from the top trachea to the deep lung, including bronchi and small bronchioles, were observed to be thoroughly covered by the bioadhesive hydrogel (FIG. 33C).

### Example 9

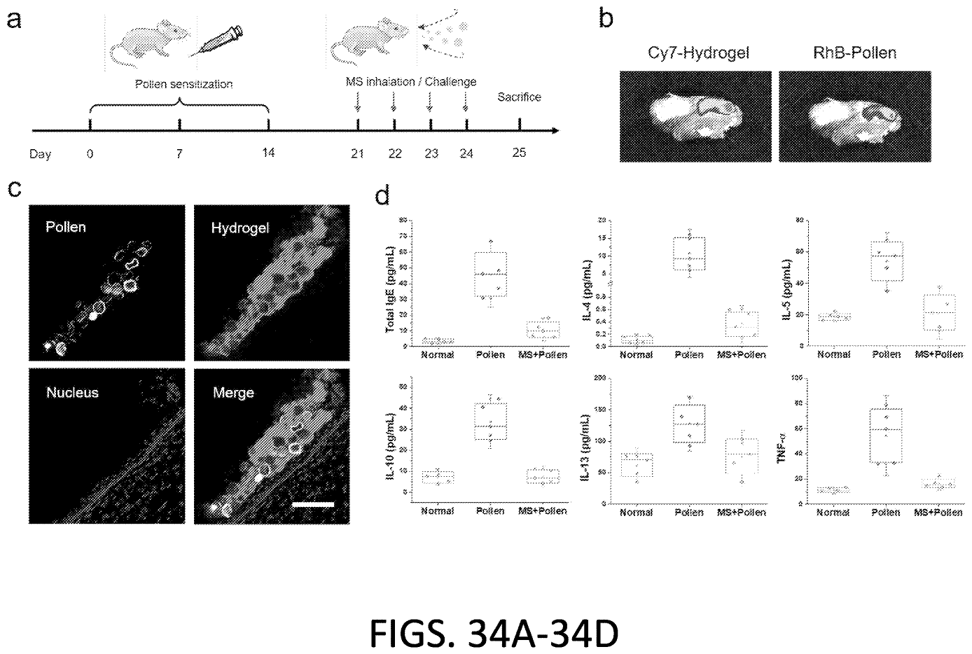

A pollen allergy mouse model was established using Balb/c mice. In brief, 8-week-old Balb/c mice underwent intraperitoneal injection with a mixture of Alum immunologic adjuvant and aspen tree pollen solution weekly for 2 weeks (3 times in total). One week after the last dose (Day 21), the mice received bioadhesive microsphere inhalation 10 minutes prior to pollen exposure for four consecutive days (FIG. 34A). IVIS imaging of the mice revealed thorough coverage of the nasal cavity with bioadhesive hydrogel, with subsequently exposed pollen particles colocalized with the hydrogel (FIG. 34B). Confocal fluorescent images of the nasal cavity illustrated that pollen particles were attached and mixed with the hydrogel formed on the nasal cilia surface (FIG. 34C). Blood samples were collected to measure serum IgE and cytokine levels (FIG. 34D). Total serum IgE levels significantly increased after pollen exposure, whereas mice pre-treated with bioadhesive microspheres showed normal IgE levels. Key cytokines (IL-4, IL-5, IL-10, IL-13, and TNF-α), highly associated with allergic reactions, increased in the control group but remained normal in the bioadhesive microsphere pre-inhalation mice.

### Example 10

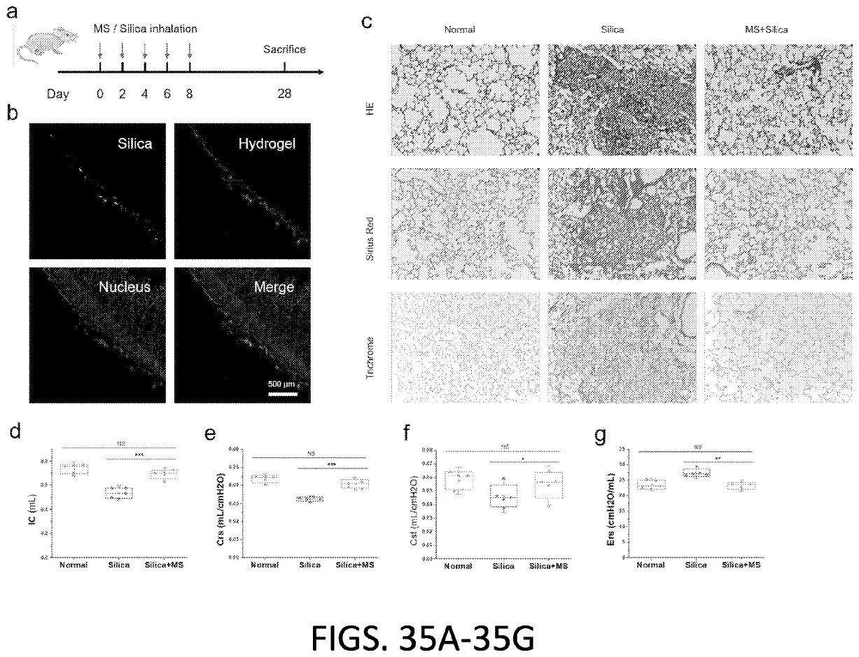

A silicosis mouse model was induced in C57/BL6 mice. In brief, bioadhesive microspheres were intratracheally administered, followed by inhalation of silica powder (1~5 μm), every other day for a total of 5 times (FIG. 35A). The mice were sacrificed on Day 28. Confocal fluorescent images of the mouse trachea demonstrated that silica particles were absorbed onto the hydrogel on the tracheal epithelium (FIG. 35B). Histology sections revealed minimal lung fibrosis in mice pre-treated with bioadhesive microspheres compared to untreated mice (FIG. 35C). Lung function was measured one day before sacrifice (FIGS. 35D-35G). The results indicated normal lung function, including pulmonary inspiratory capacity, resistance, and compliance, in comparison to the silica-treated mice.

What is claimed is:

1. An inhalable bioadhesive composition comprising:
polyacrylic acid, or a derivative thereof;
acrylic acid N-hydroxysuccinimide ester; and
a mixture of water-soluble peptides;
wherein the composition forms a cross-linked hydrogel.

2. The composition of claim 1, wherein the composition is formulated as a plurality of microparticles.

3. The composition of claim 2, wherein the plurality of microparticles are from about 0.1 μm to about 100 μm.

4. The composition of claim 2, wherein the plurality of microparticles are from about 1.0 μm to about 50 μm.

5. The composition of claim 2, wherein the plurality of microparticles have an aerodynamic diameter that is from about 0.5 μm to about 5.0 μm.

6. The composition of any one of claims 1 to 5, wherein the composition forms the cross-linked hydrogel upon exposure to lung tissue.

7. The composition of any one of claims 1 to 6, wherein the volume of the composition increases at least 5 times upon exposure to lung tissue.

8. The composition of any one of claims 1 to 7, wherein the mixture of water-soluble peptides comprises gelatin chitosan, extracellular matrix, and/or hypromellose.

9. The composition of any one of claims 1 to 8, wherein the composition is formulated as a powder.

10. The composition of any one of claims 1 to 9, wherein the water-soluble peptides are present in the composition at a concentration ranging from about 1% wt/vol to about 10% wt/vol.

11. The composition of any one of claims 1 to 10, wherein the polyacrylic acid is present in the composition at a concentration ranging from about 10% vol/vol to about 20% vol/vol.

12. The composition of any one of claims 1 to 11, wherein the acrylic acid N-hydroxysuccinimide ester is present in the composition at a concentration ranging from about 0.1% wt/vol to about 5.0% wt/vol.

13. The composition of any one of claims 1 to 12, wherein the composition further comprises a pharmaceutically acceptable carrier or excipient.

14. The composition of claim 1, wherein the composition consists essentially of polyacrylic acid, acrylic acid N-hydroxysuccinimide ester, and gelatin.

15. A method of preparing inhalable bioadhesive microparticles, the method comprising:
preparing an aqueous solution comprising polyacrylic acid, acrylic acid N-hydroxysuccinimide ester, and a mixture of water-soluble peptides;
adding the aqueous solution to an oil in a dropwise manner to generate an emulsion;
incubating the emulsion at a temperature ranging from about 10° C. to about 15° C. to generate a plurality of microparticles; and
collecting the plurality of microparticles.

16. The method of claim 15, wherein the method further comprises heating and/or agitating the aqueous solution.

17. The method of claim 15 or claim 16, wherein the oil is a food grade oil.

18. The method of any one of claims 15 to 17, wherein the plurality of microparticles are collected using filtration.

19. The method of any one of claims 15 to 18, wherein the method further comprises washing the plurality of microparticles after collection.

20. The method of any one of claims 15 to 19, wherein the method further comprises drying the plurality of microparticles.

21. The method of any one of claims 15 to 20, wherein the method further comprises formulating the plurality of microparticles into a powder.

22. The method of any one of claims 15 to 21, wherein the water-soluble peptides are present in the solution at a concentration ranging from about 10% wt/vol to about 10% wt/vol.

23. The method of any one of claims 15 to 22, wherein the polyacrylic acid is present in the solution at a concentration ranging from about 10% vol/vol to about 20% vol/vol.

24. The method of any one of claims 15 to 23, wherein the acrylic acid N-hydroxysuccinimide ester is present in the solution at a concentration ranging from about 0.1% wt/vol to about 5.0% wt/vol.

25. A method of reducing the likelihood of a pulmonary condition in a subject, the method comprising administering the composition of any one of claims 1 to 14 to the subject.

26. The method of claim 25, wherein the composition is administered as a powder using a dry powder inhalation device.

27. The method of claim 26, wherein the composition forms a cross-linked hydrogel upon exposure to lung tissue, thereby forming a barrier that reduces the likelihood of the pulmonary condition in the subject.

28. The method of any one of claims 25 to 27, wherein the composition is administered at a dose ranging from about 1-5 mg per kg of the subject's body weight.

29. The method of any one of claims 25 to 28, wherein the pulmonary condition comprises a viral infection.

30. The method of claim 29, wherein the virus is a respiratory virus.

31. The method of claim 30, wherein the respiratory virus is selected from the group consisting of an adenovirus, a coronavirus, an influenza virus, a parainfluenza virus, a parvovirus, a respiratory syncytial virus, or a rhinovirus.

32. The method of any one of claims 25 to 31, wherein reducing the likelihood of the viral infection comprises reducing infection rate.

33. The method of any one of claims 25 to 32, wherein the composition reduces the likelihood of the pulmonary condition for at least 8 hours.

34. The method of any one of claims 25 to 33, wherein the composition is administered prophylactically.

35. The method of any one of claims 25 to 28, wherein the pulmonary condition is caused by exposure to an environmental pollutant or contaminant.

36. The method of any one of claims 25 to 28, wherein the pulmonary condition is caused by an allergen.
